(19) United States
(12) Patent Application Publication
(10) Pub. No.: US 2026/0077156 A1
(43) Pub. Date: Mar. 19, 2026

(54) CATHETER METHOD OF MANUFACTURE

(71) Applicant: Piraeus Medical, Inc., Rochester, MN (US)

(72) Inventor: Jared Hutar, Cromwell, MN (US)

(21) Appl. No.: 19/330,085

(22) Filed: Sep. 16, 2025

Related U.S. Application Data
(60) Provisional application No. 63/695,206, filed on Sep. 16, 2024.

(51) Int. Cl. A61M 25/00

(52) U.S. Cl. CPC A61M 25/0009; A61M 25/0045

(57) ABSTRACT

The present invention relates to a method for coating the lumen of a tube using fluid displacement techniques. In some embodiments, the method involves introducing a first coating solution into the lumen of the tube using a positive or negative displacement, draining the excess solution to leave a film on the inner surface, and curing the film to form a uniform coating. In some embodiments, the displacement system can include a piston pump, peristaltic pump, and/or syringe. In some embodiments, a first coating solution may comprise a mixture of polyaziridine and either polyurethane or polyacrylate, while a second coating solution may comprise polysaccharide and/or polyacrylic acid and may be hydrophilic. Additional steps may include applying multiple layers, coating both the inner and outer surfaces of the tube, and/or using gas to drain the coating solution. In some embodiments, multiple tubes can be coated simultaneously using a manifold assembly.

### CROSS-REFERENCE TO RELATED APPLICATIONS

This application claims the benefit of and priority to U.S. Provisional Application No. 63/695,206, filed Sep. 16, 2024, which is hereby incorporated herein by reference in its entirety for all purposes.

### BACKGROUND

Tubes used in medical applications often require internal coatings to reduce friction or provide other functional properties. Traditional methods for coating the inner diameter of these tubes typically involve drawing coating solutions into the lumen using vacuum techniques. These methods may face limitations in terms of material waste, control, consistency, scalability, and achieving uniform coating thickness. Additionally, vacuum-based methods can introduce bubbles or other imperfections during the coating process. A need exists for a coating method that offers improved control over the application of the coating solution and delivers consistent results with greater simplicity and scalability.

### SUMMARY

Some embodiments described herein are directed to a system and methods for coating the lumen of a tube using positive displacement to introduce the first coating and second coating solution into the lumen. Some embodiments provide precise and accurate control over the volume of coating solution applied, ensuring uniform and consistent coating of the inner surface of the tube, which is particularly suitable for applications requiring a smooth, low-friction surface inside the tube, such as medical catheters and similar devices. In accordance with some embodiments, the system includes one or more of the following components and the methods comprise one or more of the following steps:

In some embodiments, a method for coating the lumen of a tube includes a step of introducing a first coating solution into the lumen of the tube using a positive displacement system. Some embodiments include a step of draining the excess first coating solution from the lumen. Some embodiments include a step of leaving a film of the first coating solution on the inner surface of the tube after it is drained. Some embodiments include a step of curing the first coating solution to form a uniform coating on the lumen of the tube.

Some embodiments include a step of introducing a second coating solution into the lumen of the tube using a positive displacement system. Some embodiments include a step of draining the excess second coating solution from the lumen. Some embodiments include a step of leaving a film of second coating solution on the inner surface of the tube. Some embodiments include a step of and curing the second coating solution to form a uniform coating on the lumen of the tube.

Some embodiments comprise moving fluid into a catheter using positive displacement. In some embodiments, the positive displacement is provided by one or more of a piston pump(s), peristaltic pump(s), and/or syringe(s).

In some embodiments, a method further comprises applying a second coating layer to the lumen using the same positive displacement system. In some embodiments, the method comprises providing a first coating solution comprising a mixture of polyaziridine, polyurethane and/or polyacrylate. According to some embodiments, the method comprises the second coating solution comprising polysaccharide and/or polyacrylic acid. According to some embodiments, the method comprises the second coating solution being hydrophilic.

In some embodiments, the method further comprises coating the inner and outer surface of the tube using the positively displaced coating delivered through the inner diameter of the tube to fill a container surrounding the tube before removing the tube with the inner and outer surface being coated.

In some embodiments, the method comprises the first coating solution being drained from the lumen by flowing a gas through the lumen. Some embodiments include a step of flowing a cleaning solution through the tube. In some embodiments, the method comprises the first coating solution further comprising a short chain alcohol. In some embodiments, the method comprises the first coating solution additionally comprising a zirconate or titanate compound.

The foregoing, and other features and advantages of the system, will be apparent from the following detailed description according to some embodiments.

### BRIEF DESCRIPTION OF THE DRAWINGS

These and other features, aspects, and advantages according to some embodiments are described below with reference to the drawings, which are intended to illustrate, but not to limit, the system and methods of the disclosure. In the drawings, like characters denote corresponding features consistently throughout some embodiments.

FIG. 1 illustrates a perspective view of a catheter system including an outer catheter and an inner catheter, according to some embodiments.

FIGS. 2A and 2B illustrate a catheter system including an outer catheter and an inner catheter, according to some embodiments.

FIG. 3 illustrates a cross-section of an outer catheter, according to some embodiments.

FIG. 4 illustrates a cross-section of an inner catheter, according to some embodiments.

FIG. 5 illustrates a cross-section of a catheter system, including an outer catheter and an inner catheter, according to some embodiments.

FIG. 6 illustrates an outer catheter, according to some embodiments.

FIG. 7 illustrates an inner catheter, according to some embodiments.

FIG. 8 illustrates an outer catheter including various elements, according to some embodiments.

FIG. 9 illustrates a catheter system including an outer catheter and an inner catheter, according to some embodiments.

FIG. 10 illustrates an inner catheter including a hypotube, according to some embodiments.

FIG. 11 illustrates an inner catheter including a coil structure, according to some embodiments.

FIG. 12 illustrates an inner catheter including a braid structure, according to some embodiments.

FIGS. 13A and 13B illustrate cross-sectional views of a hypotube, according to some embodiments.

FIG. 14 illustrates a catheter system including an outer catheter and an inner catheter, according to some embodiments.

FIG. 15 illustrates an inner catheter comprising a hypotube, according to some embodiments.

FIGS. 16A and 16B illustrate cross-sectional views of a hypotube, according to some embodiments.

FIGS. 17 and 18 illustrate flow charts explaining the processes of applying hydrophilic coatings, according to some embodiments.

FIG. 19 shows results for a 3-point bending test performed on conventional catheters and system catheters according to some embodiments.

FIG. 20 illustrates a catheter kink test for a system versus a conventional catheter according to some embodiments.

FIG. 21 shows data for inner diameter lubricity in a simulated anatomy model according to some embodiments.

FIG. 22 shows a measured catheter distal looped compression force according to some embodiments.

FIG. 23 shows a cross-sectional view of the catheter 300, according to some embodiments.

FIG. 24 shows a schematic diagram of the positive displacement coating system according to some embodiments.

FIG. 25 illustrates the positive displacement coating system of FIG. 24 attached to a manifold configuration according to some embodiments.

### DETAILED DESCRIPTION

Some embodiments described herein are directed to systems and methods for coating the lumen of a tube using positive displacement to introduce a first coating, second coating, and/or multiple additional coating solutions into the lumen. Some embodiments provide precise and accurate control over the volume of coating solution applied, ensuring uniform and consistent coating of the inner surface of the tube, which is particularly suitable for applications requiring a smooth, low-friction surface inside the tube, such as medical catheters and other devices. In accordance with some embodiments, the systems include one or more of the components described herein, and the methods comprise one or more of the steps described herein in relation to any configuration presented in the figures or written description.

Although some embodiments and examples are disclosed below, the subject matter extends beyond the some disclosed embodiments to other alternative embodiments and/or uses, and to modifications and equivalents thereof. Thus, the scope of the claims appended hereto is not limited to some embodiments described below. For example, in any method or process disclosed herein, the acts or operations of the method or process may be performed in any suitable sequence and are not necessarily limited to any particular disclosed sequence according to some embodiments. Various operations may be described as multiple discrete operations in turn, in a manner that may be helpful in understanding some embodiments; however, the order of description should not be construed to imply that these operations are order-dependent. Additionally, the structures, systems, and/or devices described herein according to some embodiments may be embodied as integrated components or as separate components.

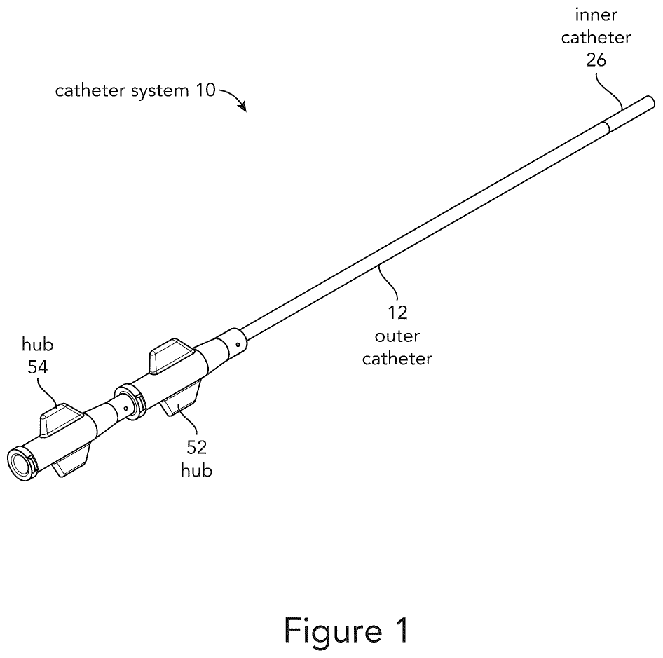

FIG. 1 illustrates a perspective view of a catheter system 10 suitable for the coating system and method described herein, according to some embodiments. In some embodiments, the catheter system includes an outer catheter 12 and an inner catheter 26, as illustrated in FIG. 1. In some embodiments, the outer catheter 12 includes a hub 52, and the inner catheter 26 includes a hub 54. The catheter system 10 will be described in greater detail throughout this disclosure.

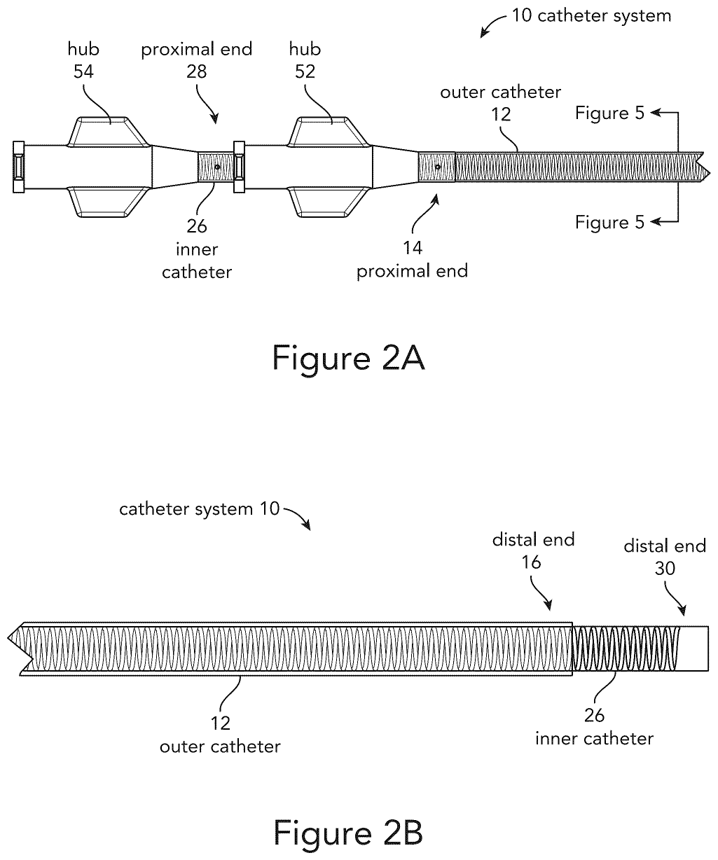

FIGS. 2A and 2B further illustrate the catheter system 10, according to some embodiments. In some embodiments, the catheter system includes an outer catheter 12 and an inner catheter 26, as illustrated in FIGS. 2A and 2B. In some embodiments, the outer catheter 12 includes a hub 52, and the inner catheter 26 includes a hub 54. As shown in FIG. 2A, the hub 52 may be located at the proximal end 14 of the outer catheter 12, and the hub 54 may be located at the proximal end 28 of the inner catheter 26. In some embodiments, the outer catheter 12 includes a distal end 16 located opposite the proximal end 14, and the inner catheter 26 includes a distal end 30 located opposite the proximal end 28. In some embodiments, the outer catheter 12 and/or inner catheter 26 includes an outer surface that is smoother than an inner surface.

In some embodiments, the outer catheter 12 may be sized and configured to at least partially receive the inner catheter 26, as illustrated in FIGS. 2A and 2B. In some embodiments, the outer catheter 12 may also be sized and configured to receive one or more traversing structures, such as a guidewire, a microcatheter, an intermediate catheter, and/or a stent retriever, to name a few non-limiting examples. In some embodiments, the catheter system 10 includes a combination of an inner and outer device (i.e., the inner and outer catheters 26, 12) that can be used concentrically, with the inner device inside of the outer device. In some embodiments, the outer and/or inner catheters 12, 26 can be used individually. For example, during a procedure, such as a thrombectomy, the outer catheter 12 may be inserted into the patient first in an initial attempt to track the outer catheter 12 distally within the anatomy to a surface of a clot. If the outer catheter 12 successfully tracks the surface of the clot, an aspiration force may be applied to the outer catheter 12, thereby removing the clot through the outer catheter 12. In some embodiments, if the outer catheter 12 is unsuccessful in tracking to the surface of the clot, the outer catheter 12 may still serve as a guide or support catheter to help deliver the inner catheter 26 through the outer catheter 12 to the surface of the clot.

In some embodiments, a method of using only a single device (i.e., the outer catheter 12) to remove the clot allows for procedures to be more efficient than current procedure practices, which often involve several steps of introducing and removing several devices. In some embodiments, the catheter system 10, including the inner catheter 26 and outer catheter 12, allows for patient anatomy to drive the procedure, rather than following the same steps for every patient, as is the current practice.

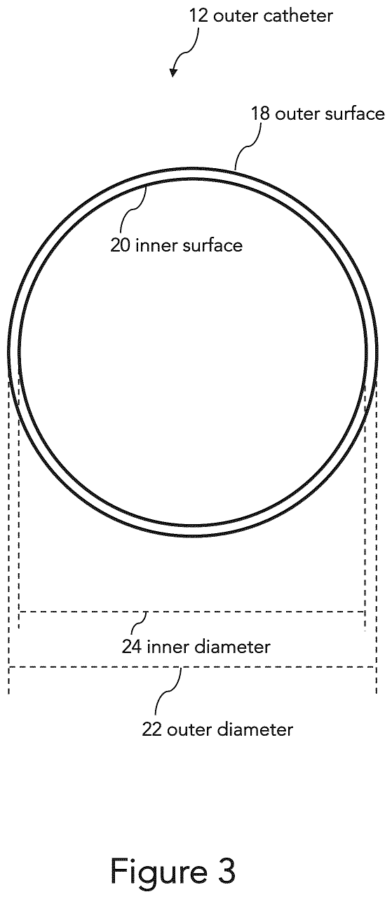

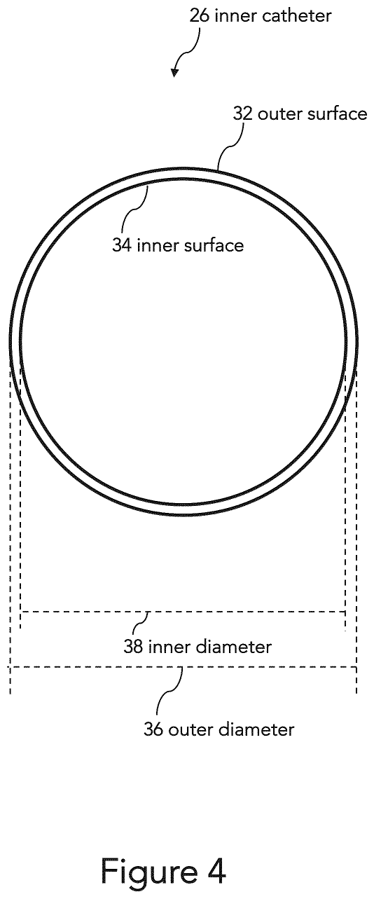

FIGS. 3 and 4 illustrate cross-section views of the outer catheter 12 and inner catheter 26, respectively. In some embodiments, the outer catheter 12 may include an outer surface 18 defining an outer diameter 22 and an inner surface 20 defining an inner diameter 24. The outer diameter 22 and inner diameter 24 may each define a broad range of dimensions, including, for example, 0.111 inches for the outer diameter 22 and 0.100 inches for the inner diameter 24, according to some embodiments. As illustrated in FIG. 4, in some embodiments, the inner catheter 26 may also include an outer surface 32 defining an outer diameter 36 and an inner surface 34 defining an inner diameter 38. In some embodiments, the outer diameter 36 of the outer surface 32 defines a measurement of 0.098 inches, and the inner diameter 38 of the inner surface 34 defines a measurement of 0.088 inches, although any inner and/or outer measurement can be used, as the dimensions listed here, for both the outer catheter 12 and the inner catheter 26, are included as non-limiting examples. In some embodiments, the outer catheter 12 and inner catheter 26 may both define a wide range of dimensions not explicitly listed in this disclosure. For example, the outer catheter 12 and inner catheter 26 may define outer and/or inner diameter dimensions between 0.003 inches and 0.18 inches, according to some embodiments.

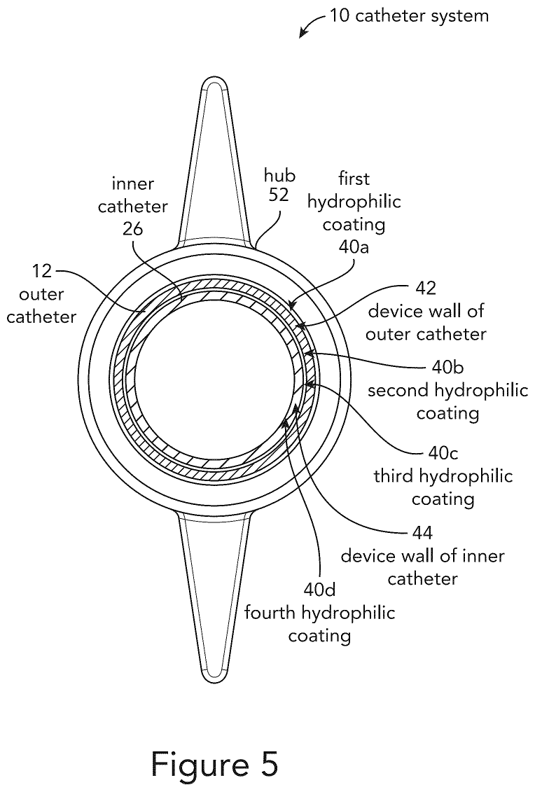

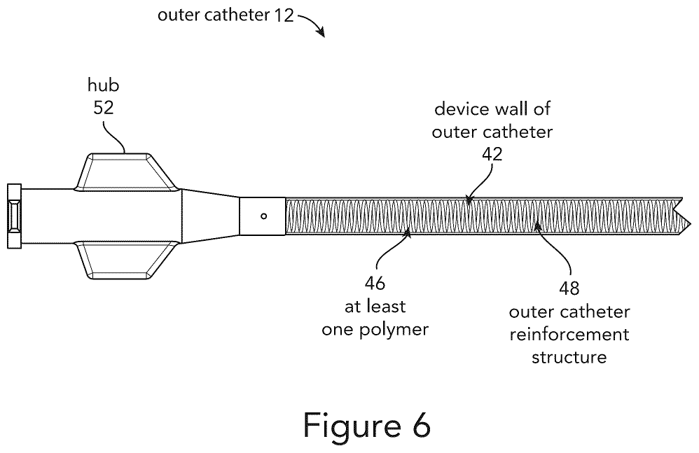

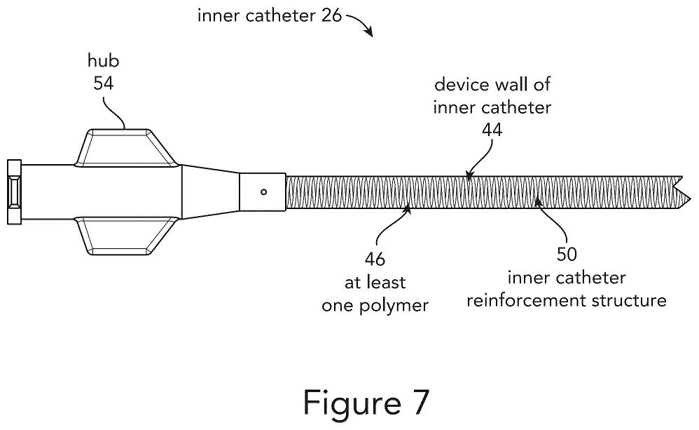

As indicated in FIG. 2A, FIG. 5 illustrates a cross-section of the catheter system 10, including both the outer catheter 12 and inner catheter 26, as well as the hub 52 of the outer catheter 12 according to some embodiments. In some embodiments, as shown in FIG. 5, the outer catheter 12 comprises a device wall of the outer catheter 42, and the inner catheter 26 comprises a device wall of the inner catheter 44. The device walls 42, 44 will be discussed further with reference to FIGS. 6 and 7. In some embodiments, the catheter system 10 may also include a first hydrophilic coating 40a, a second hydrophilic coating 40b, a third hydrophilic coating 40c, and a fourth hydrophilic coating 40d, as shown in FIG. 5.

In some embodiments, the first hydrophilic coating 40a is located on the outer surface 18 of the outer catheter 12, and the second hydrophilic coating 40b is located on the inner surface 20 of the outer catheter 12. In some embodiments, the device wall of the outer catheter 42 is located between the first hydrophilic coating 40a and the second hydrophilic coating 40b. In some embodiments, the third hydrophilic coating 40c is located on the outer surface 32 of the inner catheter 26, and/or the fourth hydrophilic coating 40d is located on the inner surface 34 of the inner catheter 26. In some embodiments, the device wall of the inner catheter 44 is located between the third hydrophilic coating 40c and the fourth hydrophilic coating 40d. In some embodiments, each of the first, second, third, and fourth hydrophilic coatings 40a-d may extend along a surface extending between the proximal ends 14, 28 and distal ends 16, 30 of the outer and inner catheters 12, 26. In some embodiments, the surface extends substantially an entire length of the catheters 12, 26. In some embodiments, the surface may extend less than a full length, such as 50%, 25%, or 10% of the entire length. In some embodiments, each of the hydrophilic coatings 40a-d is configured to cover a distalmost portion, such as 15 centimeters, of the outer and inner catheters 12, 26. It should be noted that, in some embodiments each of the hydrophilic coatings 40a-d is configured to cover any size portion of the catheters 12, 26. It should also be noted that each of the hydrophilic coatings 40a-d do not necessarily define the same length, though they may each define the same length according to some embodiments.

In some embodiments, each of the first hydrophilic coating 40a, second hydrophilic coating 40b, third hydrophilic coating 40c, and fourth hydrophilic coating 40d may comprise the same material and thickness. In some embodiments, the thickness of each hydrophilic coating 40a-d is between 0.0001 and 0.001 inches. The term “hydrophilic coating” is a species of lubricious coatings that reduce friction and increases trackability of the outer and inner catheters 12, 26, as they move within vessels and/or within one another (e.g., the inner catheter 26 moving within the outer catheter 12) according to some embodiments. Some nonlimiting examples of lubricious coatings according to some embodiments include hydrophilic coatings, silicone coatings, PTFE dust, and any other suitable lubricants. In some embodiments, coating the device wall 42, 44 with one or more lubricious coatings (e.g., hydrophilic coating 40a-d) allows the device walls 42, 44 to be thinner than traditional device walls while also improving the performance of the catheters 12, 26. In some embodiments, the device walls 42, 44 may include a thickness between 0.001 and 0.04 inches.

Referring now to FIG. 6, the outer catheter 12 is shown, as previously discussed, the outer catheter 12 includes a device wall 42 according to some embodiments. In some embodiments, as illustrated in FIG. 6, the device wall 42 comprises at least one polymer 46 and an outer catheter reinforcement structure 48. In some embodiments, the at least one polymer 46 may also be referred to as a “polymer jacket structure.” In some embodiments, the at least one polymer 46 is configured to provide at least one of flexibility and structural support to the outer catheter 12 and/or the inner catheter 26. In some embodiments, the outer catheter reinforcement structure 48 may comprise a (metallic) braid and/or coil structure, with the at least one polymer 46 filling any space within the braid and/or coil structure. In some embodiments, the at least one polymer 46 may also cover the outer catheter reinforcement structure 48. In some embodiments, the outer catheter reinforcement structure 48 is configured to provide stiffness to a proximal portion of the outer catheter 12 and flexibility to a distal portion of the outer catheter 12. In some embodiments, the amount, coil tightness, and/or composition of the outer catheter reinforcement structure 48 and/or inner catheter 26 may vary depending on the location along the length of the outer catheter 12 and/or length of inner catheter 26.

Similar to FIG. 6, FIG. 7 shows the inner catheter 26 including the device wall 44 comprising the at least one polymer 46 and the inner catheter reinforcement structure 50 according to some embodiments. In some embodiments, the inner catheter reinforcement structure 50 is substantially similar in construction to the outer catheter reinforcement structure 48, including the structure immersed in the at least one polymer 46. In some embodiments, the inner catheter reinforcement structure 50 may comprises one or both of a coil and braded structure. In some embodiments, the inner catheter reinforcement structure 50 is configured to provide stiffness to a proximal portion of the inner catheter 26, and flexibility to a distal portion of the inner catheter 26. In some embodiments, the amount, coil tightness, and/or composition of the inner catheter reinforcement structure 50 may vary depending on the location along the length of the inner catheter 26. In some embodiments, the device wall of the outer catheter 42 and the device wall of the inner catheter 44 is substantially the same construction and may comprise the same type of polymer(s) in the at least one polymer 46, as well as the same type of braid and/or coil structure in the outer catheter reinforcement structure 48 and/or inner catheter reinforcement structure 50. In some embodiments, the inner catheter 26 may be considered a scaled-down version of the outer catheter 12, with the same elements but smaller dimensions.

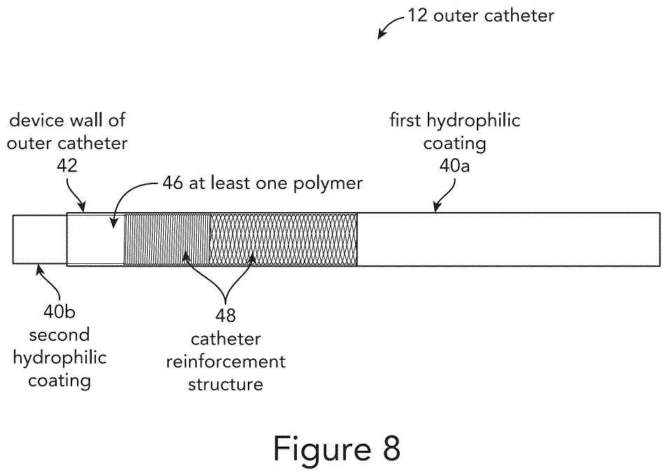

FIG. 8 is also similar to FIG. 6 in that it illustrates another view of the outer catheter 12, including the various layers of materials according to some embodiments. Included in FIG. 8 are the first hydrophilic coating 40a, the second hydrophilic coating 40b, the device wall 42, the at least one polymer 46, and the outer catheter reinforcement structure 48. In some embodiments, the device wall 42 may have a structure where the at least one polymer 46 and the outer catheter reinforcement structure 48 are melded together, with the first hydrophilic coating 40a located on the outer surface and the second hydrophilic coating 40b located on the inner surface.

In some embodiments, the device wall 42 has a “sandwich” structure comprising two layers of the at least one polymer 46 directly coupled together, with the outer catheter reinforcement structure 48 between the polymer layers 46. As previously discussed, the outer catheter reinforcement structure 48 may comprise a braid and/or coil structure. In some embodiments, the outer catheter reinforcement structure 48 and/or inner catheter reinforcement structure 50 may each include individual coil and/or braid structures, as indicated by the different appearances of the outer catheter reinforcement structure 48 in FIG. 8. For example, the coil structure is represented by the portion of the outer catheter reinforcement structure 48 to the left in FIG. 8, while the braid structure is represented by the portion of the outer catheter reinforcement structure 48 to the right in FIG. 8 according to some embodiments. In some embodiments, the “sandwich” style device wall 42 comprises an inner layer of at least one polymer 46, the coil structure on top of the inner polymer layer, the braid structure on top of the coil structure, and an outer layer of at least one polymer 46. In some embodiments, in the “sandwich” style, the device wall 42 also includes the first hydrophilic coating 40a and the second hydrophilic coating 40b.

In some embodiments, the “sandwich” style device wall 42 allows for a larger (more open) coil pitch in the coil structure, thereby enabling the outer catheter 12 to be softer than some embodiments where the coil structure has a tighter or more closed pitch. In some embodiments, a softer and more flexible outer catheter 12 can be desirable for certain uses, such as when navigating tortuous anatomy, to give the user (i.e., a medical practitioner) more freedom to move the device at different angles. In some embodiments, this “sandwich” style provides benefits from a manufacturing standpoint, as a more open coil pitch is easier to produce and may include a larger margin of error than a closed pitch. Some embodiments described herein are directed to a method of manufacture of the inner and/or outer catheters.

However, there are benefits to a device wall 42 comprising a tighter pitch coil structure. For example, in some embodiments, the outer catheter reinforcement structure 48 includes a coil defining a pitch smaller than 0.03 inches, and/or the second hydrophilic coating 40b is provided with a substantially solid and/or ribbed surface to adhere to. In this sense, the second hydrophilic coating 40b (as well as the fourth hydrophilic coating 40d of the inner catheter 26) may be thought of as having a textured, or “ribbed,” surface according to some embodiments. In comparison, in some embodiments, the first hydrophilic coating 40a (and the third hydrophilic coating 40c) may be thought of as having a substantially smooth surface. In some embodiments, the combination of textured and smooth surfaces of the hydrophilic coatings 40a-d provide just enough friction to allow a user to easily control movement of the outer catheter 12 and the inner catheter 26. For example, when the second hydrophilic coating 40b has a textured surface and the third hydrophilic coating 40c has a smooth surface, there may be enough friction between the two surfaces to prevent excessive and difficult-to-control sliding of the inner catheter 26 within the outer catheter 12, as may be the case if both hydrophilic coatings 40b, 40c were smooth.

In some embodiments, to ensure a sufficiently solid inner surface 20 of the outer catheter 12, the coil comprises a 0.002 inch round coil with a 0.004 inch pitch. In some embodiments, a tighter pitch coil may be better for facilitating lubricity of the inner surface 20 of the outer catheter 12. In some embodiments, a coil with a pitch less than 0.025 inches is desirable. In some embodiments, a sufficiently tight-pitch coil in the outer catheter reinforcement structure 48, combined with the second hydrophilic coating 40b on the inner surface 20 of the outer catheter 12, provides enough lubricity to replace the need for a liner, such as a PTFE liner, which is traditionally used in catheter construction. In some embodiments, the coil may comprise a round coil, a flat coil, and/or other types of coil design.

Regardless of the “style” of device wall 42 used (e.g., “sandwich” or tight-pitch coil), the use of a first and second hydrophilic coating 40a, 40b on the outer catheter 12 may allow for a thinner, more flexible device wall 42, as compared to other types of catheter walls without inner and outer coatings according to some embodiments. It should be noted that though FIG. 8 specifically labels the catheter as the outer catheter 12, the layers shown in FIG. 8 and the preceding discussion also apply to the inner catheter 26 in some embodiments, such that FIG. 8 may be considered as depicting either the outer catheter 12 or the inner catheter 26.

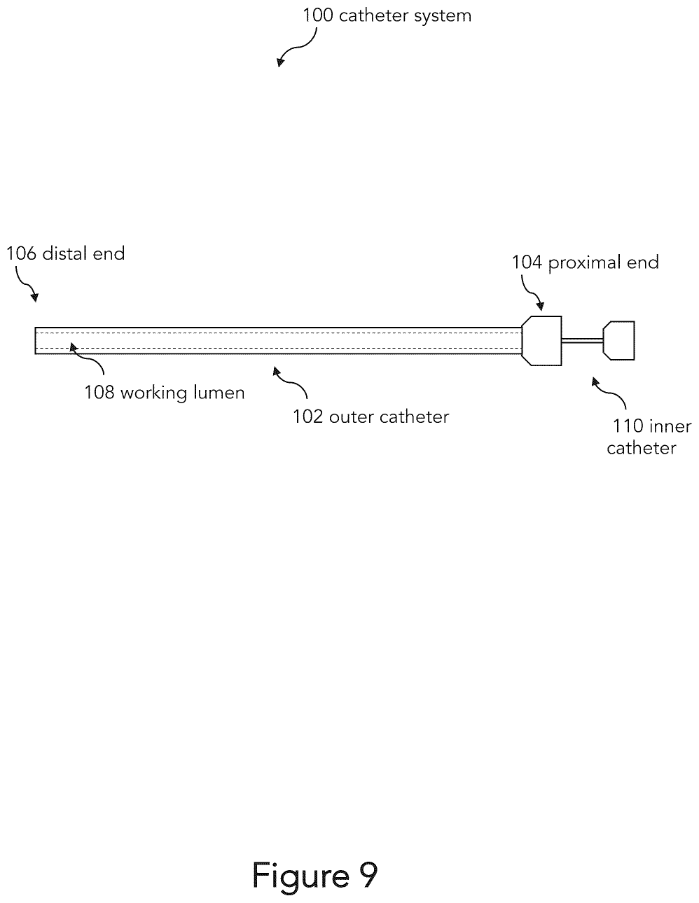

FIG. 9 illustrates a catheter system 100 comprising an outer catheter 102 and an inner catheter 110, which may be substantially similar in construction to outer catheter 12 and/or inner catheter 26 according to some embodiments. As shown, the outer catheter 102 may include a proximal end 104 and a distal end 106 located opposite the proximal end 104. In some embodiments, the outer catheter 102 includes a working lumen 108 extending between the proximal end 104 and the distal end 106. In some embodiments, the working lumen 108 includes the space inside the catheter 110 configured to allow the passage of various instruments, such as guidewires, microcatheters, stent retrievers, and/or is used for the aspiration of clots and other obstructions within a patient's vasculature. In some embodiments, the working lumen 108 is configured to at least partially receive the inner catheter 110.

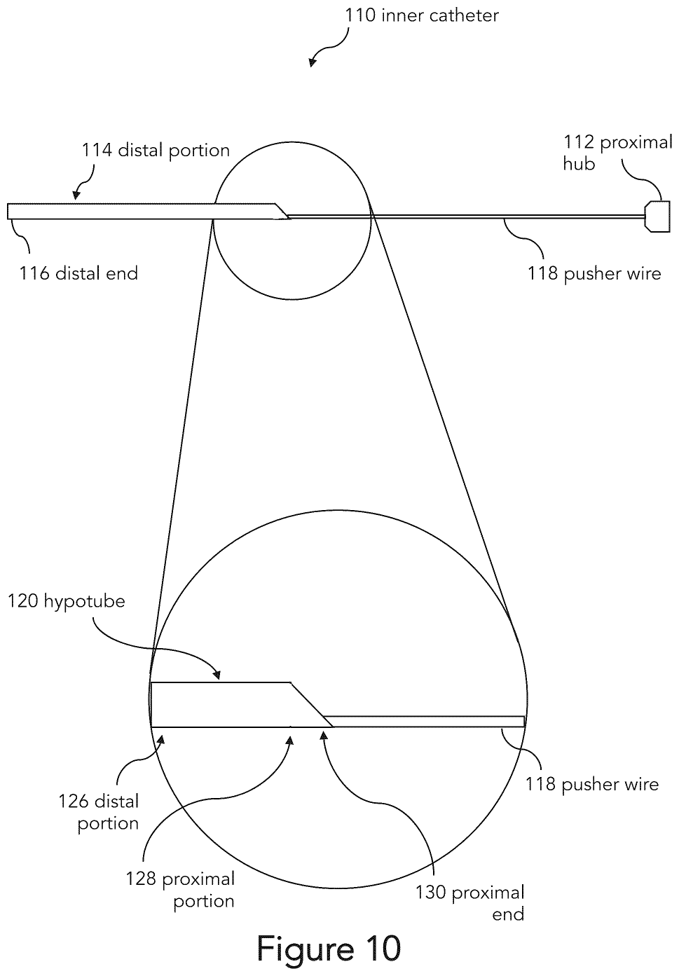

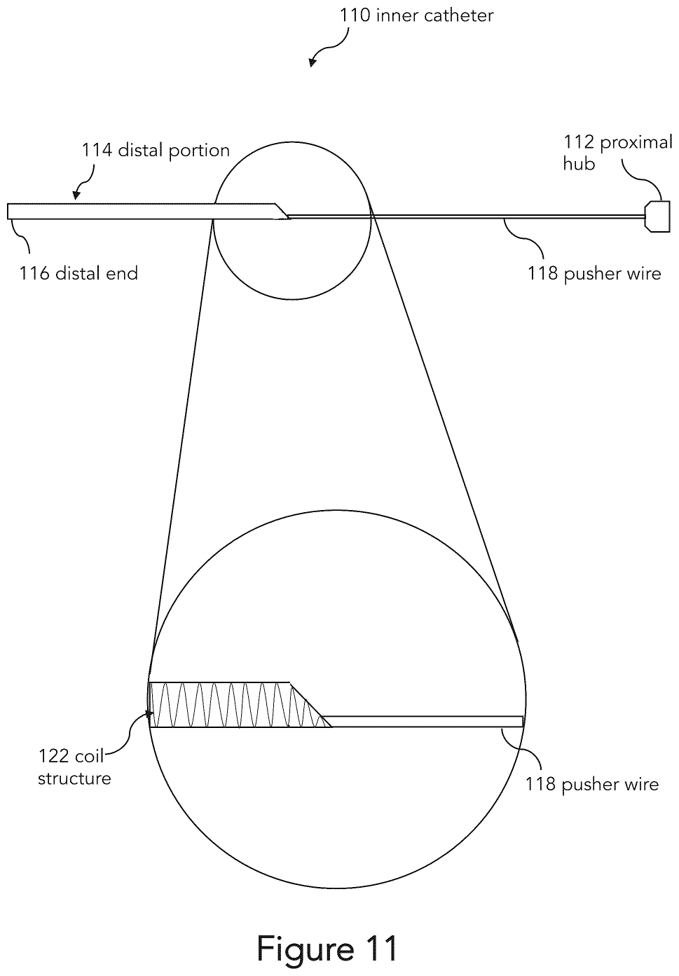

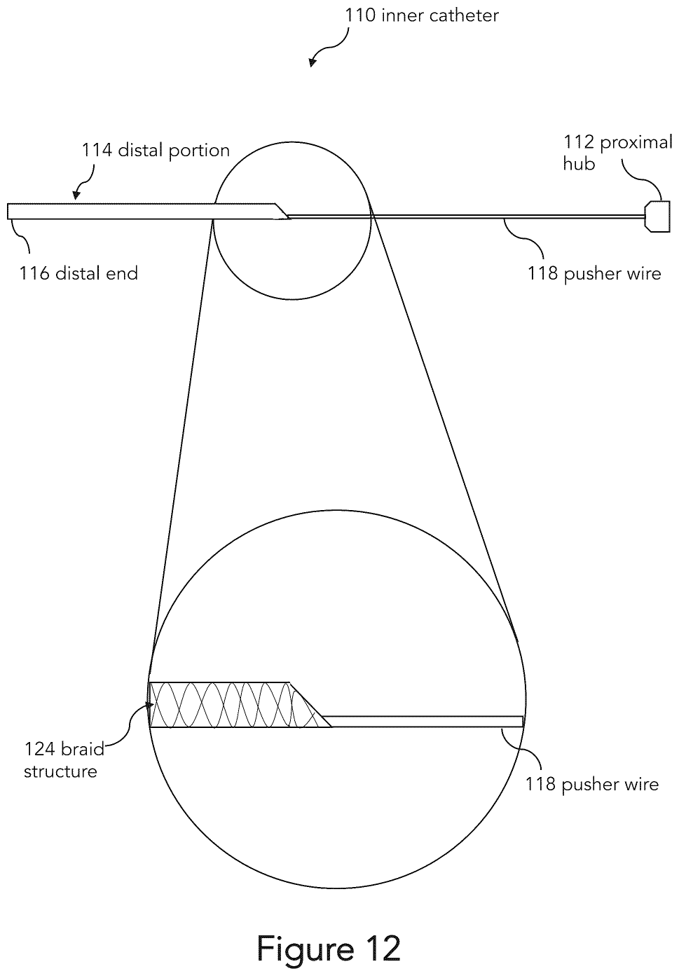

FIGS. 10-12 illustrate the inner catheter 110, which, in some embodiments, comprises a proximal hub 112, a distal portion 114 having a distal end 116 located opposite the proximal hub 112, and a pusher wire 118 extending between the proximal hub 112 and the distal portion 114. In some embodiments, the working lumen 108 is configured to at least partially receive the pusher wire 118, as shown in FIG. 9. As illustrated in the inset view of FIG. 10, in some embodiments, the distal portion 114 may comprise a hypotube 120. In some embodiments, the hypotube 120 comprises a stainless steel hypotube. In some embodiments, the hypotube 120 may comprise a nitinol hypotube. In some embodiments, the hypotube 120 is comprised of any suitable material, and, in some embodiments, is a laser-cut hypotube. In some embodiments, the hypotube 120 may comprise a non-laser-cut hypotube. In some embodiments, the hypotube 120 comprises a distal portion 126 and a proximal portion 128 located opposite the distal portion 126. As shown, the proximal portion 128 is configured to taper to a proximal end 130 coupled to the pusher wire 118. Rather than a hypotube 120, the distal portion 114 of the inner catheter 110 may comprise a coil structure 122, as illustrated in FIG. 11, and/or a braid structure 124, as illustrated in FIG. 12 according to some embodiments. In some embodiments, the distal portion 114 of the inner catheter 110 comprises a combination of the coil structure 122 and the braid structure 124. In some embodiments, the distal portion 114 may comprise any suitable material configuration and is not limited to the examples shown in the Figures and discussed in this disclosure.

As shown in FIGS. 10-12, and due to the inclusion of the pusher wire 118, in some embodiments, the distal portion 114 of the inner catheter 110 includes a length substantially less than the full length of the inner catheter 110. In comparison, the outer catheter 102 shown in FIG. 9 may comprise a single tube defining substantially the full length of the outer catheter 102, minus the proximal end 104 according to some embodiments. In some embodiments, in the catheter system 100, the outer catheter 102 may be considered a “full catheter” and the inner catheter 110 may be considered a “partial catheter.” In some embodiments, the distal portion 114 of the inner catheter 110, whether a hypotube 120, coil structure 122, and/or braid structure 124, may include a length of about twenty centimeters. In some embodiments, the distal portion 114 defines a length less than twenty centimeters. In some embodiments, the distal portion 114 may define a length greater than twenty centimeters.

In some embodiments, the pusher wire 118 is fixedly coupled (e.g., via welding, bonding, adhesive, or the like) to the distal portion 114 of the inner catheter 110. In some embodiments, the pusher wire 118 is configured to facilitate navigation of the inner catheter 110 through the working lumen 108 of the outer catheter 102. For example, according to a method of use, during a procedure, a physician (or another qualified medical professional) is configured to “push” the inner catheter 110 through the outer catheter 102 using the proximal hub 112 and/or the pusher wire 118 according to some embodiments. In some embodiments, the relative rigidity of the pusher wire 118 may help advance the inner catheter 110 with limited twisting, kinking, bending, etc. of the distal portion 114. In some embodiments, the pusher wire 118 comprises a round wire. In some embodiments, the pusher wire 118 may comprise a flat (or any other desired shape) wire.

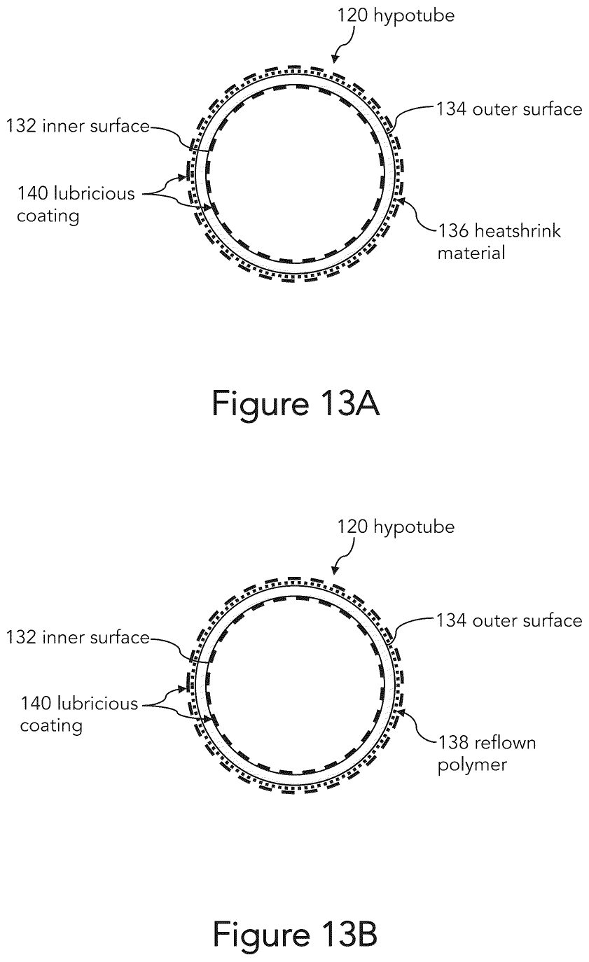

Turning now to FIGS. 13A and 13B, cross-sectional views of the hypotube 120 according to some embodiments are shown. In some embodiments, the hypotube 120 comprises an inner surface 132 and an outer surface 134 located opposite the inner surface 132. In some embodiments, the outer surface 134 is covered in a heatshrink material 136, as shown in FIG. 13A. In some embodiments, the heatshrink material 136 comprises a material laminated, fused, and/or melted onto the outer surface 134 of the hypotube 120. In some embodiments, at least a portion of the heatshrink material 136 and at least a portion of the inner surface 132 of the hypotube 120 are coated with a lubricious coating 140. In some embodiments, substantially the entirety of the heatshrink material 136 and substantially the entirety of the inner surface 132 are coated with the lubricious coating 140. In some embodiments, at least a portion of the heatshrink material 136 and substantially the entirety of the inner surface 132 is coated with the lubricious coating 140. In some embodiments, substantially the entirety of the heatshrink material 136 and at least a portion of the inner surface 132 is coated with the lubricious coating 140. In some embodiments, the inner surface 132 varies in diameter along the length of the catheter.

In some embodiments, the lubricious coating 140 may comprise a hydrophilic coating. In some embodiments, the lubricious coating 140 comprises silicone. In some embodiments, the lubricious coating 140 may comprise any suitable type of coating, and is not intended to be limited to the examples discussed in this disclosure. In some embodiments, the lubricious coating 140 helps facilitate smooth navigation of the hypotube 120 through the working lumen 108 of the outer catheter 102. In some embodiments, where the inner catheter 110 extends distally from the outer catheter 102, the lubricious coating 140 may also help facilitate smooth navigation of the hypotube 120 through a patient's vasculature. In some embodiments, the lubricious coating 140 on the inner surface 132 of the hypotube 120 facilitates smooth movement of a secondary device (e.g., a guidewire, microcatheter, specialized device, etc.) through the hypotube 120.

FIG. 13B illustrates that, in some embodiments, the outer surface 134 of the hypotube 120 is covered in a reflown polymer 138 rather than a heatshrink material 136 according to some embodiments. In some embodiments, at least a portion of the reflown polymer 138 and at least a portion of the inner surface 132 of the hypotube 120 is coated with the lubricious coating 140. In some embodiments, substantially the entirety of the reflown polymer 138 and substantially the entirety of the inner surface 132 of the hypotube 120 are coated with the lubricious coating 140. In some embodiments, at least a portion of the reflown polymer 138 and substantially the entirety of the inner surface 132 is coated with the lubricious coating 140. In some embodiments, substantially the entirety of the reflown polymer 138 and at least a portion of the inner surface 132 is coated with the lubricious coating 140.

In some embodiments, the outer surface 134 of the hypotube 120 is covered with a combination of the heatshrink material 136 and the reflown polymer 138. In some embodiments, at least a portion of the hypotube 120 includes a PTFE liner rather than the lubricious coating 140. For example, in some embodiments, half of the hypotube 120 may include a PTFE liner while the other half includes the lubricious coating 140. In some embodiments, half of the hypotube 120 may include a PTFE liner while the other half includes no lubricious coating 140. In some embodiments, the hypotube 120 may also include neither a PTFE liner nor a lubricious coating 140. In some embodiments, the catheter system 100 including the coil structure 122 and/or braid structure 124, as illustrated in FIGS. 11 and 12, respectively, may also include a heatshrink material 136, reflown polymer 138, or combination thereof to cover the coil structure 122 and/or braid structure 124, as applicable. In some embodiments, the coil structure 122 and/or braid structure 124 may include the lubricious coating 140 as illustrated in FIGS. 13A and 13B.

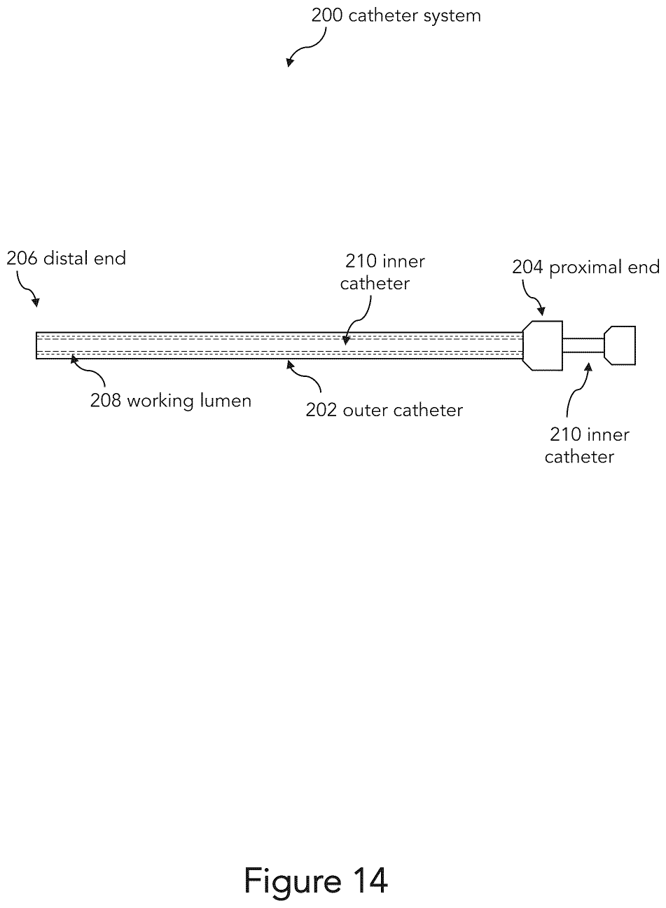

Referring now to FIG. 14, a catheter system 200 is shown according to some embodiments. In some embodiments, the catheter system 200 comprises an outer catheter 202 having a proximal end 204, a distal end 206 located opposite the proximal end 204, and a working lumen 208 extending between the proximal end 204 and the distal end 206. In some embodiments, the catheter system 200 may also include an inner catheter 210, and the working lumen 208 is configured to at least partially receive the inner catheter 210, as demonstrated in FIG. 14. As with any catheter described herein according to some embodiments, the outer catheter 202 and inner catheter 210 may be made by similar manufacturing methods and/or comprise similar structures, such as those described in relation to FIGS. 6-8 and 23.

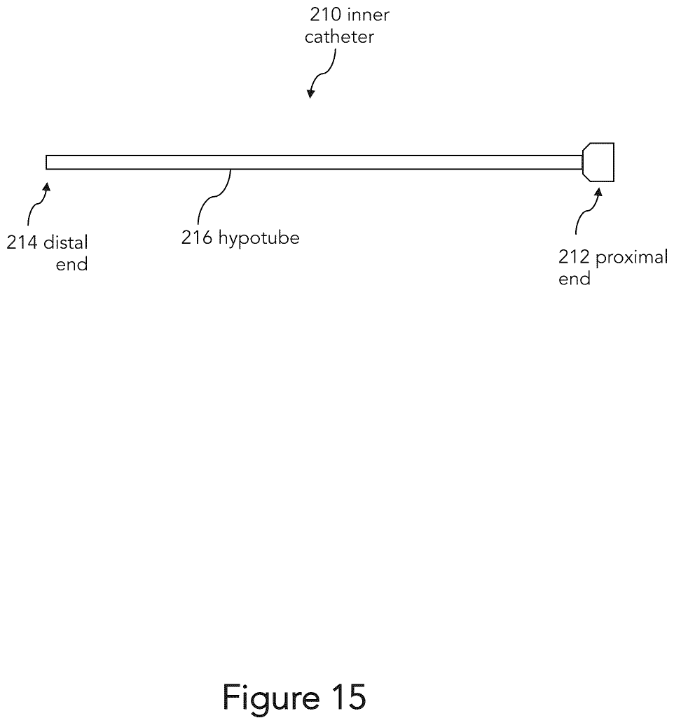

FIG. 15 illustrates the inner catheter 210 in more detail, including the proximal end 212 and the distal end 214 located opposite the proximal end 212. Unlike the inner catheter 110 of the catheter system 100 (shown in FIGS. 9-13) according to some embodiments, the inner catheter 210 may comprise a “full” catheter rather than a “partial” catheter. Stated differently, in some embodiments, the inner catheter 210 may comprise a hypotube 216 configured to extend the full length from the proximal end 212 to the distal end 214. Similar to the hypotube 120 of the inner catheter 110, in some embodiments, the hypotube 216 of the inner catheter 210 may comprise a laser-cut hypotube. In some embodiments, the inner catheter 210 may comprise a non-laser-cut hypotube. In some embodiments, the hypotube 216 comprises a stainless steel hypotube. In some embodiments, the hypotube 216 may comprise a nitinol hypotube, or a hypotube constructed of any other suitable material.

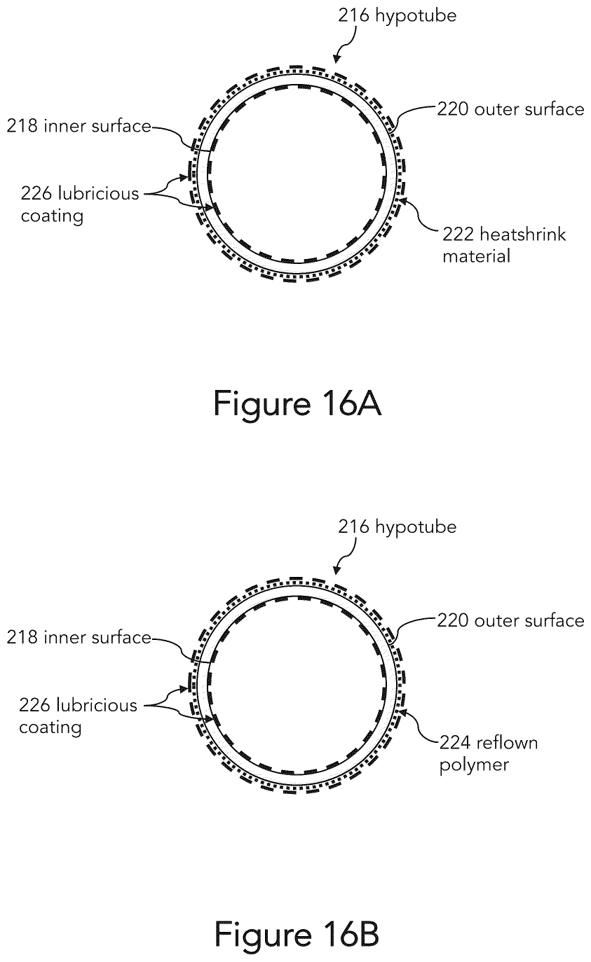

FIGS. 16A and 16B are similar to FIGS. 13A and 13B, though they illustrate the hypotube 216 rather than the hypotube 120 according to some embodiments. FIGS. 16A and 16B show cross-sectional views of the hypotube 216, wherein, in some embodiments, the hypotube 216 comprises an inner surface 218 and an outer surface 220 located opposite the inner surface 218. In some embodiments, the outer surface 220 is covered in a heatshrink material 222, as shown in FIG. 16A. In some embodiments, the heatshrink material 222 may comprise a material laminated or melted onto the outer surface 220 of the hypotube 216. In some embodiments, at least a portion of the heatshrink material 222 and at least a portion of the inner surface 218 of the hypotube 216 are coated with a lubricious coating 226. In some embodiments, substantially the entirety of the heatshrink material 222 and substantially the entirety of the inner surface 218 are coated with the lubricious coating 226. In some embodiments, at least a portion of the heatshrink material 222 and substantially the entirety of the inner surface 218 is coated with the lubricious coating 226. In some embodiments, substantially the entirety of the heatshrink material 222 and at least a portion of the inner surface 218 is coated with the lubricious coating 226.

In some embodiments, the lubricious coating 226 is substantially similar to the lubricious coating 140 of the catheter system 100. In some embodiments, the lubricious coating 226 may comprise a hydrophilic coating. In some embodiments, the lubricious coating 226 comprises silicone. In some embodiments, the lubricious coating 226 may comprise any suitable type of coating, and is not intended to be limited to the examples discussed in this disclosure. In some embodiments, the lubricious coating 226 helps facilitate smooth navigation of the hypotube 216 through the working lumen 208 of the outer catheter 202. In some embodiments, where the inner catheter 210 extends distally from the outer catheter 202, the lubricious coating 226 may also help facilitate smooth navigation of the hypotube 216 through a patient's vasculature. In some embodiments, the lubricious coating 226 on the inner surface 218 of the hypotube 216 facilitates smooth movement of a secondary device (e.g., a guidewire, microcatheter, specialized device, etc.) through the hypotube 216, similar to some embodiments described herein.

FIG. 16B illustrates that, in some embodiments, the outer surface 220 of the hypotube 216 is covered in a reflown polymer 224 rather than a heatshrink material 222. In some embodiments, at least a portion of the reflown polymer 224 and at least a portion of the inner surface 218 of the hypotube 216 is coated with the lubricious coating 226. In some embodiments, substantially the entirety of the reflown polymer 224 and substantially the entirety of the inner surface 218 of the hypotube 216 are coated with the lubricious coating 226. In some embodiments, at least a portion of the reflown polymer 224 and substantially the entirety of the inner surface 218 is coated with the lubricious coating 226. In some embodiments, substantially the entirety of the reflown polymer 224 and at least a portion of the inner surface 218 is coated with the lubricious coating 226.

In some embodiments, the outer surface 220 of the hypotube 216 is covered with a combination of the heatshrink material 222 and the reflown polymer 224. In some embodiments, at least a portion of the hypotube 216 includes a PTFE liner rather than the lubricious coating 226. For example, in some embodiments, half of the hypotube 216 may include a PTFE liner while the other half includes the lubricious coating 226. In some embodiments, half of the hypotube 216 may include a PTFE liner while the other half includes no lubricious coating 226. In some embodiments, the hypotube 216 may also include neither a PTFE liner nor a lubricious coating 226. In some embodiments, the inner catheter 210 may comprise, rather than the hypotube 216, a coil structure and/or braid structure, similar to those illustrated in FIGS. 6, 7, 8, 11 and 12. In some embodiments, the inner catheter 210 comprising a coil and/or braid structure also includes a heatshrink material 222, reflown polymer 224, or combination thereof to cover the coil structure and/or braid structure, as applicable. In addition, embodiments with the coil structure and/or braid structure may include the lubricious coating 226 as illustrated in FIGS. 16A and 16B.

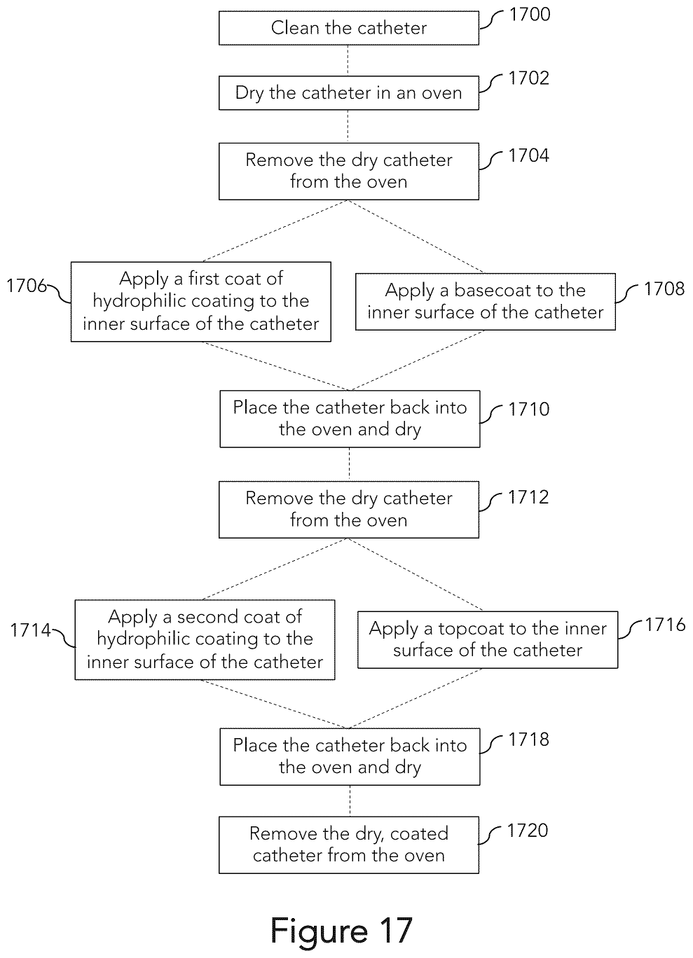

FIG. 17 shows a flowchart illustrating a nonlimiting example process of coating and curing an inner surface of a catheter according to some embodiments. For the purposes of this disclosure, the “catheter” recited in FIG. 17 may comprise elements of the catheter system 10 (i.e., the outer catheter 12 or the inner catheter 26), elements of the catheter system 100 (i.e., the outer catheter 102 or the inner catheter 110), and/or elements of the catheter system 200 (i.e., the outer catheter 202 or the inner catheter 210). The steps of the process, which include a method of manufacture, should be considered as applying to any of the catheters recited in this disclosure.

In some embodiments, the process shown in FIG. 17 starts with cleaning the catheter, at step 1700. In some embodiments, cleaning the catheter includes flushing the catheter with purified water, isopropyl alcohol (“IPA”), a mix of IPA and water, and/or some other suitable cleansing fluid. In some embodiments, the next step is to dry the catheter in an oven, at step 1702. In some embodiments, the drying step may include placing the clean catheter in an oven set to a temperature between 0° C. and 400° C. and applying positive or negative pressured air (e.g., oxygen, a mix of oxygen and nitrogen, etc.) to the hub of the catheter in order to dry the inner surface of the catheter according to some embodiments. In some embodiments, the process continues with step 1704: remove the dry catheter from the oven.

Next, in some embodiments, the process can continue in one of two possible steps. One option is to apply a first coat of hydrophilic coating to the inner surface of the catheter, shown at step 1706 according to some embodiments. In some embodiments, a basecoat is applied to the inner surface of the catheter, at step 1708. Both steps 1706 and 1708 may use positive or negative pressure to fill the catheter with either the hydrophilic coating (step 1706) or the basecoat (step 1708) according to some embodiments. In some embodiments, the catheter is filled with the relevant coating material from either end of the catheter body. In some embodiments, the relevant coating material substantially continuously flows through the catheter for a predetermined amount of time to ensure an adequate amount of coating is applied. In some embodiments, the relevant coating material may dwell within the catheter, rather than flow through, for a predetermined amount of time.

After either step 1706 or step 1708, in some embodiments the process may continue to place the catheter back into the oven to dry, at step 1710. Similar to the first drying step (i.e., step 1702), in some embodiments step 1710 may involve placing the clean catheter in an oven set to a temperature between 0° C. and 400° C. and applying positive or negative pressured air (e.g., oxygen, a mix of oxygen and nitrogen, etc.) to the hub of the catheter in order to dry the inner surface of the catheter. Step 1710 is considered a “heat curing” step, as heat is used to dry (i.e., cure) the coating according to some embodiments. Next, in some embodiments, the positive or negative pressure source is disconnected and the dry catheter is removed from the oven, at step 1712.

At this point, in some embodiments, the process again diverges into two different options. In some embodiments, one option is to apply a second coat of hydrophilic coating to the inner surface of the catheter, at step 1714. In some embodiments, the other option is to apply a topcoat to the inner surface of the catheter, at step 1716. Similar to the application of the first coat of hydrophilic coating (at step 1706) and the application of the basecoat (at step 1708), in some embodiments, both steps 1714 and 1716 may use positive or negative pressure to fill the catheter with the relevant coating material from either end of the catheter body. In some embodiments, the relevant coating material substantially continuously flows through the catheter for a predetermined amount of time to ensure an adequate amount of coating is applied. In some embodiments, the relevant coating material may dwell within the catheter, rather than flow through, for a predetermined amount of time.

Next, in some embodiments, the process continues with placing the catheter back into the oven to dry (or “heat cure”) again, at step 1718. Like the first and second drying steps (step 1702 and step 1710), in some embodiments, step 1718 may involve placing the catheter in an oven set to a temperature between 0° C. and 400° C. and/or applying positive or negative pressured air (e.g., oxygen, a mix of oxygen and nitrogen, etc.) to the hub of the catheter in order to dry the inner surface of the catheter. In some embodiments, the process concludes by disconnecting the positive or negative pressure source and removing the dry, coated catheter from the oven, at step 1720.

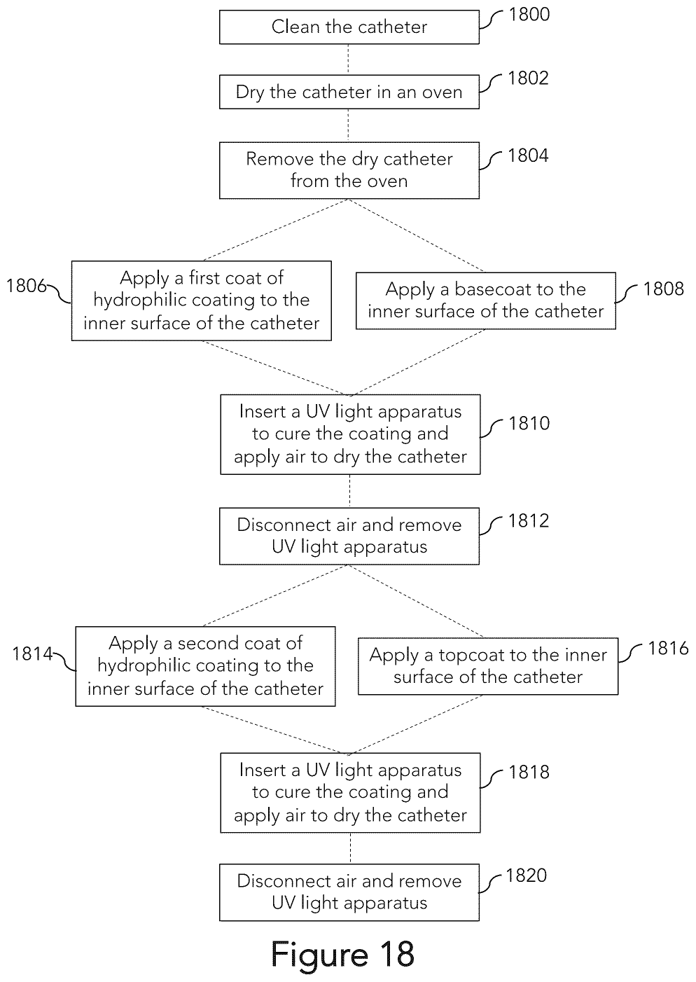

FIG. 18 is similar to FIG. 17, and includes a flowchart illustrating a slightly different nonlimiting example process of coating and curing an inner surface of a catheter according to some embodiments. As with the process shown in FIG. 17, for the purposes of this disclosure, the “catheter” recited in FIG. 18 may comprise elements of the catheter system 10 (i.e., the outer catheter 12 or the inner catheter 26), elements of the catheter system 100 (i.e., the outer catheter 102 or the inner catheter 110), elements of the catheter system 200 (i.e., the outer catheter 202 or the inner catheter 210), and/or catheter 300. The steps of the process should be considered as applying to any of the catheters recited in this disclosure according to some embodiments.

In some embodiments, the process shown in FIG. 18 starts with cleaning the catheter, at step 1800. In some embodiments, cleaning the catheter includes flushing the catheter with purified water, IPA, a mix of IPA and water, or some other suitable cleansing fluid. In some embodiments, the next step is to dry the catheter in an oven, at step 1802. The drying step may include placing the clean catheter in an oven set to a temperature between 0° C. and 400° C. and applying positive or negative pressured air (e.g., oxygen, a mix of oxygen and nitrogen, etc.) to the hub of the catheter in order to dry the inner surface of the catheter according to some embodiments. In some embodiments, the process continues with step 1804: removing the dry catheter from the oven.

Next, in some embodiments, the process can continue in one of two possible steps. In some embodiments, one option is to apply a first coat of hydrophilic coating to the inner surface of the catheter, shown at step 1806. In some embodiments, a basecoat is applied to the inner surface of the catheter, at step 1808. In some embodiments, both steps 1806 and 1808 may use positive or negative pressure to fill the catheter with either the hydrophilic coating (step 1806) or the basecoat (step 1808). In some embodiments, the catheter is filled with the relevant coating material from either end of the catheter body. In some embodiments, the relevant coating material substantially continuously flows through the catheter for a predetermined amount of time to ensure an adequate amount of coating is applied. In some embodiments, the relevant coating material may dwell within the catheter, rather than flow through, for a predetermined amount of time. In some embodiments, a reduction in flow is used to increase the dwell time within a catheter.

After either step 1806 or step 1808, in some embodiments, the process may continue by inserting a UV light apparatus to cure the coating and applying positive or negative pressured air (e.g., oxygen, a mix of oxygen and nitrogen, etc.) to the hub of the catheter in order to dry the inner surface of the catheter, at step 1810. In some embodiments, the UV light apparatus is inserted into the inner diameter of the catheter to cure the coating on the inner surface. Next, the positive or negative pressure source is disconnected and the UV light apparatus is removed from the catheter, at step 1812, according to some embodiments.

At this point, in some embodiments, the process again diverges into two different options. In some embodiments, one options is to apply a second coat of hydrophilic coating to the inner surface of the catheter, at step 1814. In some embodiments, the other option is to apply a topcoat to the inner surface of the catheter, at step 1816. Similar to the application of the first coat of hydrophilic coating (at step 1806) and the application of the basecoat (at step 1808), in some embodiments, both steps 1814 and 1816 may use positive or negative pressure to fill the catheter with the relevant coating material from either end of the catheter body. In some embodiments, the relevant coating material substantially continuously flows through the catheter for a predetermined amount of time to ensure an adequate amount of coating is applied. In some embodiments, the relevant coating material may dwell within the catheter, rather than flow through, for a predetermined amount of time.

Next, in some embodiments, the process continues with another round of UV light curing, at step 1818. Like the first UV curing step (step 1810), step 1818 may involve inserting a UV light apparatus to cure the coating and applying positive or negative pressured air (e.g., oxygen, a mix of oxygen and nitrogen, etc.) to the hub of the catheter in order to dry the inner surface of the catheter. In some embodiments, the UV light apparatus is inserted into the inner diameter of the catheter to cure the coating on the inner surface. In some embodiments, the process includes disconnecting the positive or negative pressure source and removing the UV light apparatus from the catheter, at step 1820.

The catheter system 10 is configured for use in various procedures conducted in a variety of locations of a patient's anatomy. Though brain-specific thrombectomy is discussed, the disclosure should not be considered limiting to any specific type or location of the procedure. The catheter system 10 is used for the aspiration of clots throughout a patient's body, and the various aspects of the catheter system 10 discussed above may improve the rate of clot removal in a number of procedure locations.

Catheter systems may include a full outer catheter 102 and partial inner catheter 110, like the catheter system 100, or may include a full outer catheter 202 and a full inner catheter 210, like the catheter system 200. In some embodiments, a catheter system includes a partial outer catheter and a full inner catheter. In some embodiments, a catheter system may also include a partial outer catheter and a partial inner catheter.

Though not shown in the figures, a method of using any of the catheter systems described herein according to some embodiments, such as the catheter system 10, the catheter system 100, the catheter system 200, and/or catheter shaft 300 comprises inserting an outer catheter, such as the outer catheter 12, the outer catheter 102, and/or the outer catheter 202, into a patient's vasculature, wherein the outer catheter includes a proximal end and a distal end located opposite the proximal end, advancing the outer catheter through the patient's vasculature toward a vascular lesion, and advancing the outer catheter to a location selected from the group consisting of a first location and a second location. In some embodiments, the first location is within a first predetermined distance from the vascular lesion, and the second location is within a second predetermined distance from the vascular lesion. In some embodiments, when the outer catheter is in the first location, the outer catheter is able to aspirate the vascular lesion, and when the outer catheter is in the second location, the outer catheter is unable to aspirate the vascular lesion. In some embodiments, when the outer catheter is in the first location, the method further comprises aspirating the vascular lesion with the outer catheter. In some embodiments, when the outer catheter is in the second location, the method may further comprise advancing an inner catheter, such as the inner catheter 26, the inner catheter 110, and/or the inner catheter 210, through the outer catheter toward the first location. In some embodiments, when the inner catheter is in the first location, the method further comprises aspirating the vascular lesion with the inner catheter.

In some embodiments, a fluoropolymer-free manufacturing process for the catheter begins by selecting a PTFE mandrel that matches the desired inner diameter of the catheter. In some embodiments, one or more reinforcement structures are placed on and/or over the mandrel. In some embodiments, a reinforcement structure includes a wire, string, coil, and/or laser cut hypotube. In some embodiments, different reinforcement structures are placed in different regions of the mandrel and/or are overlaid on each other. In some embodiments, the reinforcement structure includes one or more of a flat surface, a round surface, or some combination of the two.

In some embodiments, the reinforcement structure includes a coil structure, which may include elements such as one or more flat wires and/or one or more round wires as discussed above. While some embodiments may describe the use of one element type (e.g., wire, string) or element shape (e.g., round, flat), it is understood that the specific elements and/or element shapes are interchangeable when describing the metes and bounds of the system, and reference to an “element” is a reference to any combination of element types and/or shapes described herein.

In some embodiments, to form the coil structure, a wire is wound over the polytetrafluoroethylene (PTFE) mandrel using a chosen element, shape, and/or pitch, after which the coil structure is terminated. In some embodiments, the coil structure is configured to leave recesses in the inner diameter. In some embodiments, the coil structure is configured to impart a rib pattern comprising peaks and valleys on the inner diameter of the catheter.

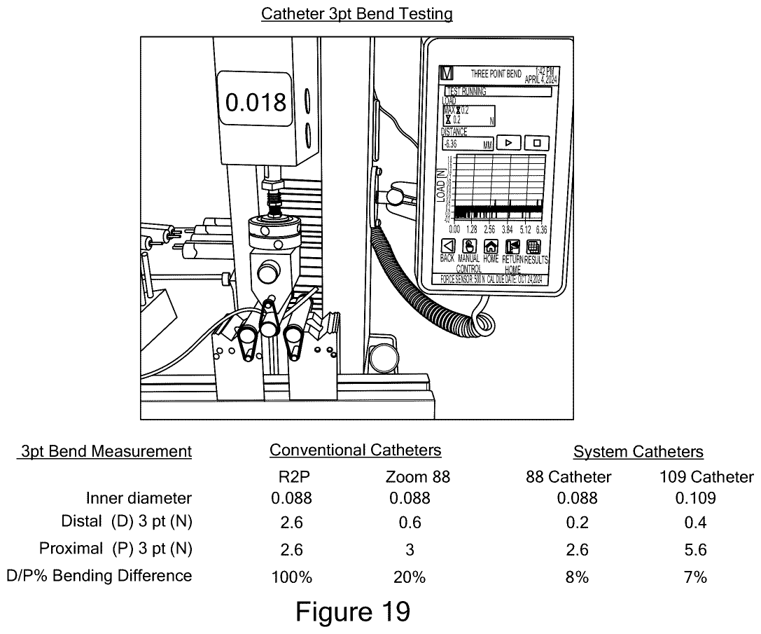

In some embodiments, a braid is then applied over the coiled PTFE mandrel, utilizing the selected material, braid pattern, and braid picks per inch (PPI) to obtain a desired structural integrity. However, it has been found that the coil structure alone, in accordance with some embodiments, provides sufficient properties to achieve the results shown in FIGS. 19-22.

In some embodiments, the reinforcement structure is wound around the mandrel in a braided pattern. In some embodiments, the braided pattern is configured to leave recesses in the inner diameter. A non-limiting recess shape includes a polygon shape, which may include a diamond or square pattern and/or grid pattern according to some embodiments. Braiding patterns formed using Steeger USA® machines have been found to produce acceptable results and provide a variety of patterns including flat braids, square braids, spiral braids, strands, and coils.

In some embodiments, the recess shape for any reinforcement structure described herein is configured to create peaks and valleys along the inner diameter of the catheter, where the valleys are configured to reduce the contact area of the inner diameter by 15-85%. In some embodiments, the peaks define the inner diameter contact area for a substantially smooth portion of a traversing structure (e.g., guide wire). In some embodiments, the peaks include a flat surface, such as in the case of a flat wire coil structure and/or hypotubes. Flat wires may increase stiffness and/or compressive strength (including vacuum strength) but may have greater contact area against a surface of a traversing structure according to some embodiments. Round wires may decrease stiffness and/or compressive strength but generate less friction for a traversing structure due to a smaller surface area at a round peak as compared to a flat peak.

In some embodiments, the reinforcement structure includes one or more hypotubes. In some embodiments, a hypotube may include one or more hollow portions. In some embodiments, the hollow portions include material removed from the hypotube in a pattern shape. In some embodiments, a step includes creating a pattern shape in the form of one or more holes longitudinal slots, spiral (helical) cuts, circular (ring) cuts, intersecting grids (e.g., mesh, crisscross lines), and/or any custom geometric pattern. In some embodiments, pattern shapes are configured to impart specific functionality such as expansion, flexibility, or kink resistance.

In some embodiments, the use of laser cutting enables the creation of precise and intricate patterns along the hypotube. Laser cutting provides clean cuts with minimal burrs and heat-affected zones, ensuring the structural integrity and smoothness of the tube in accordance with some embodiments. In some embodiments, the hypotubes is made from biocompatible metals, such as stainless steel or nickel-titanium alloys (Nitinol), which offer excellent strength and flexibility, as well as resistance to corrosion.

In some embodiments, the reinforcement structure forming step, a platinum iridium marker band is positioned over the braid. In some embodiments, the ends of the braid are trimmed flush with the marker band, and the braid is bonded to the marker band using a urethane-based adhesive. This ensures the marker band is securely attached and the transition between materials is smooth in some embodiments.

In some embodiments, a next phase includes loading each of the polymer extrusions, or tubes, over the coiled and braided reinforcement structure while on the mandrel. In some embodiments, this process starts with the stiffest polymer (e.g., ML24) and concludes with the softest (e.g., 42A Neusoft), which is placed adjacent to the marker band. This step, in some embodiments, creates a gradient of flexibility along the length of the catheter, providing both reinforcement and pliability where needed.

In some embodiments, an expanded fluorinated ethylene propylene (FEP) heat shrink is then loaded over the polymer extrusions, and the assembly is placed into a reflow machine. In some embodiments, the reflow machine employs heated forced air to melt the polymer extrusions, causing them to fuse together and bond to the metallic reinforcement structure. During this process, at least a portion of the polymer flows through the hollow portions of the reinforcement structure. In some embodiments, at least a portion of the polymer coats an inner diameter of the reinforcement structure, sealing the catheter. In some embodiments, at least a portion of the inner diameter is not coated by the polymer, and/or at least a peak surface of the inner diameter is not coated with a polymer. In some embodiments, a peak surface of the inner diameter is coated with a polymer. In some embodiments, polymer located in the area of a hollow portion creates a valley on the inner diameter of the catheter.

After the reflow process, the FEP heat shrink is removed, and the PTFE mandrel is extracted from the assembly according to some embodiments. In some embodiments, the formed catheter does not include PTFE and/or a PTFE liner along the inner diameter. Some embodiments include a step to over mold a hub onto the stiff (e.g., ML24) end of the tube, creating a secure connection point for the catheter. In some embodiments, the hub is attached to the catheter before any hydrophilic coating is applied to the catheter as described above. In some embodiments, an extended portion of polymer material is left in front of the marker band to facilitate subsequent hydrophilic coating processing, which will enhance the catheter's lubricity and ease of use during medical procedures.

In some embodiments, the catheter is then prepared for the hydrophilic coating processing by cleaning the outer diameter, which may include using a wipe saturated with a cleaning agent (e.g., 70% isopropyl alcohol (IPA)) to ensure a clean surface for coating adherence. Some embodiments include a step of coupling a fluid source (e.g., 20 cc syringe) to the catheter at the hub and flushing the catheter interior with a cleaning agent (e.g., 70% IPA) to remove any contaminants that may interfere with the coating process.

Following the flush, a hydrophilic basecoat is aspirated into the catheter using a vacuum pump (e.g., another 20 cc syringe) to coat the inner diameter of the catheter. To coat the outer diameter, the catheter is at least partially submerged into a container filled with hydrophilic fluid to a specific depth, which is carefully controlled to achieve the desired coating thickness according to some embodiments. Once the desired coating depth is reached, the vacuum is released, enabling the hydrophilic fluid to drain from the inner diameter in some embodiments. In some embodiments, the catheter is withdrawn from the hydrophilic fluid at a consistent rate to ensure an even coating. In some embodiments, the rate of withdrawal affects surface tension which determines coating thickness.

The catheter is then placed in an oven set to a curing temperature of 50-70° C. (e.g., 60° C.) for a duration of 20-40 (e.g., 30) minutes according to some embodiments. In some embodiments, an airline is connected to the hub during this time, which facilitates the curing of the hydrophilic basecoat on both the inner and outer diameters simultaneously. In some embodiments, air is slowly forced through the catheter. By adjusting the rate of air flow, in some embodiments, it is possible to control the thickness of the hydrophilic coating on the inner diameter. A slower air flow allows more time for the coating to set, potentially leading to a thicker coating, while a faster air flow thins out the coating and/or speeds up the drying process according to some embodiments.

In some embodiments, the process is repeated for a hydrophilic topcoat. In some embodiments, a 20 cc syringe is used to aspirate the topcoat into the catheter, which is then dipped into a container of hydrophilic top coat to the predetermined depth to achieve the desired coating thickness. After reaching the desired depth, in some embodiments the catheter is flushed (e.g., by releasing the vacuum and/or syringe) to remove any excess topcoat and then removed steadily.

In some embodiments, the catheter is placed back into the oven at a curing temperature of 50-70° C. (e.g., 60° C.) for a duration of 20-40 (e.g., 30) minutes. In some embodiments, the airline is still connected to the hub and/or the airline is connected to the hub. In some embodiments, air is supplied to the inner diameter at a same or similar temperature as the curing temperature, allowing the hydrophilic topcoat to cure on both the inner and outer diameters of the catheter simultaneously. In some embodiments, the sacrificial extended tip of the catheter is cut off, and the tip is rounded to ensure a smooth and/or finished end ready for medical use.

In some embodiments, the Fluoropolymer Free Manufacturing process includes constructing a catheter system that is compatible with hydrophilic coatings. In some embodiments, the catheter structure and/or manufacturing process does not include the use of fluoropolymers within the catheter and instead utilizes a range of materials that are conducive to the application of hydrophilic coatings.

In some embodiments, the catheter's reinforcement structure may include a stainless steel coil, with alternative material options such as nitinol or tungsten. As discussed previously, the reinforcement structure (e.g., coil structure) cross-section can be either round or flat in shape according to some embodiments. Similarly, the catheter may include a stainless steel braid for additional reinforcement in some embodiments, with the same alternative material options and the choice between round and/or flat types of braid. In some embodiments, as an alternative and/or in addition to using coil and/or braid components, the construction may employ a laser-cut hypotube made from Stainless Steel or Nitinol. Depending on application requirements, the catheter may feature just a coil, just a braid, or just a hypotube for reinforcement.

In some embodiments, the termination of the reinforcement structure is achieved using a urethane-based UV adhesive, ensuring a secure end. In some embodiments, the catheter's tubing transitions from rigid to soft materials, starting with Nylon tubing, specifically Vestimid ML24, which is the most rigid polymer used in catheters, followed by Vestimid ML21, the second most rigid polymer, according to some embodiments.

To create a smooth transition in flexibility, in some embodiments, PEBAX tubing is used in the middle of the device. The PEBAX material comes in varying hardness levels, denoted by the “D” hardness scale, with higher numbers indicating greater rigidity. In some embodiments, the catheter utilizes PEBAX tubing in descending order of rigidity, from 72 D to 25 D.

For the tip of the device, which requires maximum navigability, urethane tubing is employed in some embodiments. The “D” hardness scale intersects with the “A” hardness scale at 25 D, approximately equivalent to 80 A. In some embodiments, the urethane tubing used progresses from 25 D at the proximal end (i.e., adjacent to the hub) to the softer 42A Neusoft as the distal end. In some embodiments, at the very tip (distal end) of the catheter, a Platinum Iridium marker band is incorporated. This marker band allows physicians to visually confirm the catheter's tip location under fluoroscopy according to some embodiments.

In some embodiments, the catheter's female hub connector is constructed by overmolding 72 D PEBAX onto the nylon end of the device. Alternatively, a pre-molded hub is attached using UV adhesive in some embodiments. To ensure a smooth transition from the nylon shaft to the 72 D PEBAX hub, Polyolefin heat shrink is used as a strain relief in some embodiments, providing a seamless and secure connection. In some embodiments, the hydrophilic coating gets pushed through using the hub. In some embodiments, an inner diameter of the hub is also coated with a hydrophilic coating as a result of this process, further reducing friction. Conventional catheters do not include hydrophilic coating hubs as conventional manufacturing processes require catheters be coated before any hydrophilic coating is applied.

In some embodiments, the catheter includes a substantially smooth outer diameter (OD) surface. In some embodiments, the OD surface has a texture depth less than 0.002″.

In some embodiments, one or more catheters described herein include a (spiral) pattern of offset surfaces configured to enhance the lubricity of the inner diameter. In some embodiments, the contacting surface of the catheter, which interfaces with traversing devices (e.g., pusher wires, catheters) during delivery or with blood clots during aspiration, includes a width between 0.001″ and 0.004″ from one contact surface to another. In some embodiments, the contacting surface comprises materials selected from a group comprising stainless steel, Nitinol, nylon, Pebax, and Pellethane as previously described.

In some embodiments, the catheter inner diameter (ID) includes a recessed surface (i.e., valley) formed between the polymer and reinforcement structure, set back from the contacting surface by a distance (i.e., depth) ranging from 0.0001″ to 0.010″. In some embodiments, the recessed surface has a width between peaks of 0.001″ and 0.007″, where the recessed surface defines the pitch of coil structure. In some embodiments, the recessed surface includes the polymer material and/or the hydrophilic coatings previously described. In some embodiments, both the contacting and recessed surfaces are coated with a hydrophilic layer(s) that may include one or more of Polyvinylpyrrolidone (PVP), Polyacrylic acid (PAA), or Hyaluronic Acid (HA), as non-limiting examples.

In some embodiments, the resulting catheter includes a hydrophilic coating with varying thickness along its length. In some embodiments, one or more contacting surfaces include a thin hydrophilic coating with a thickness from approximately 0.0001″ to 0.000198″. In some embodiments, non-contacting areas (recessed areas) include a thick hydrophilic coating ranging from 0.0002″ to 0.003″. In some embodiments, surface tension keeps the hydrophilic coating in place while curing, where more hydrophilic coating will gather in the recessed area, resulting in an inner diameter with alternating hydrophilic coating thickness.

In some embodiments, the catheter is configured to withstand vacuum forces up to 29.92″ Hg and maintain a circular shape or not collapse more than 30% of original dimension. In some embodiments, the catheter is configured to withstand a minimum burst pressure of 300 KPA (42.5 psi) (stable up to 100 psi) for a duration of 30 seconds while not leaking and/or substantially maintaining shape. In some embodiments, the catheter's construction is configured to provide tip softness and kink resistance as further described herein.

As previously mentioned, in some embodiments, the catheter includes hydrophilic-coated hubs configured to facilitate the delivery of interventional devices and/or to minimize friction during clot removal. In some embodiments, a method of identifying a hydrophilic-coated hubs includes coloring the hub with Tantalum Blue dye to enhance visibility and identification of the hydrophilic coating process.

In some embodiments, the catheter includes a hydrophilic coated inner diameter hub, which may include polycarbonate or pvacs hubs with a hydrophilic coat. In some embodiments, the coating on the interior of the hub is configured to increase lubricity, further facilitating the delivery of devices through the catheter.

In some embodiments, the catheter's hypotubes are manufactured using laser cutting techniques to create a spiral pattern. In some embodiments, the laser cuts are of a specific size and distance apart, which promotes inner lubricity. In some embodiments, the precise laser cutting technique, combined with the hydrophilic coating, provides the necessary lubricity for the catheter's inner surfaces. In some embodiments, the offset, repeating patterns in the inner diameter of the catheter enhance lubricity and allow for the elimination of a PTFE liner.

FIG. 19 shows results for a 3-point bending test performed on conventional catheters and system catheters according to some embodiments. In some embodiments, 3-point test can be used to determine flexural strength, flexural modulus (stiffness), and/or elasticity. In some embodiments, results show stiffness characteristics for a proximal section (closer to the hub) and a distal section (closer to the end) of conventional and system catheters. In some embodiments, using the system and methods described herein, a ratio between the stiffness of the distal section and the proximal section is 15% or less, which is much less than conventional catheters. In some embodiments, a distal/proximal ratio for one or more catheters described herein is between approximately 5-10%.

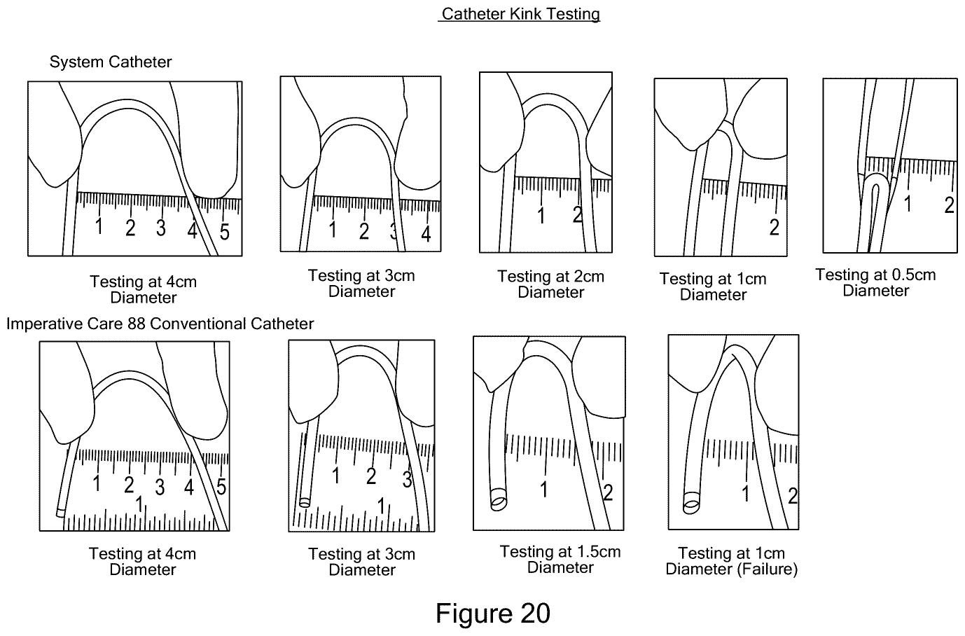

FIG. 20 illustrates a catheter kink test for a system versus a conventional catheter according to some embodiments. Kinking, in the context of this disclosure, refers to the undesirable bending, twisting, and/or creasing of a tube structure (i.e., catheter) to such an extent that it obstructs and/or blocks the motion of a fluid and/or a traversing structure moving within the tube structure. Any reference to “kinking” in this disclosure can be replaced with “collapsing” and/or “creasing” when defining the metes and bounds of the system. In addition, kinking includes a decrease in inner diameter of more than 30% along a first axis of a catheter cross-section, and/or an increase in inner diameter of more than 30% along a second axis of a catheter cross-section.

As shown in FIG. 20, conventional catheters kink and/or collapse at diameter ranges of less than 1.5 cm, where the diameter is measured from one outside portion of the catheter to the other. In contrast, in some embodiments, system catheters are configured to bend to diameter ranges less than 1.5 cm without kinking. In some embodiments, system catheters are configured to bend to diameter ranges less than 1.0 cm without kinking. In some embodiments, system catheters are configured to bend to diameter ranges less than 0.5 cm without kinking, which is far superior to any conventional catheter. In some embodiments, system catheters are configured to bend to a diameter range between 1.5 cm-5 cm.

In some embodiments, system catheters are configured to bend to an inner radius of curvature less than 16 mm without kinking. In some embodiments, system catheters are configured to bend to an inner radius of curvature less than 10 mm without kinking. In some embodiments, system catheters are configured to bend to an inner radius of curvature less than 5 mm without kinking. In some embodiments, system catheters are configured to bend to an inner radius of curvature less than 2 mm without kinking. In some embodiments, system catheters are configured to bend to an inner radius of curvature less than or equal to 1 mm without kinking, which corresponds to the 0.5 cm test shown in FIG. 20. In some embodiments, system catheters are configured to bend to an inner radius of curvature range of 15 mm-1 mm.

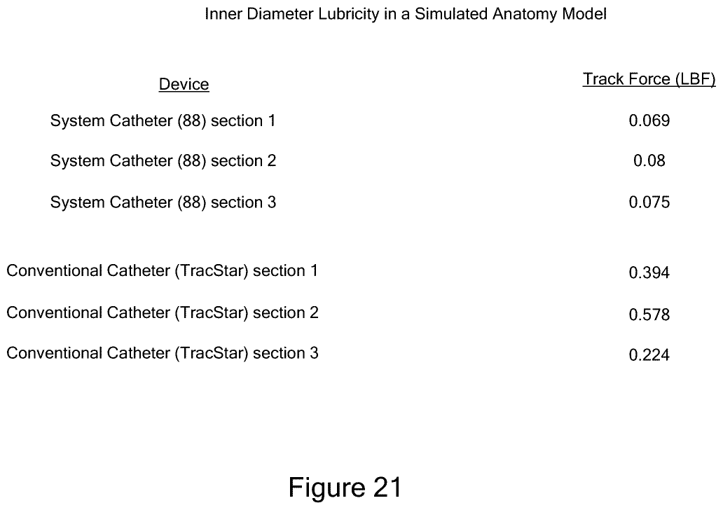

FIG. 21 shows data for inner diameter lubricity in a simulated anatomy model according to some embodiments. Inner diameter lubricity testing in a simulated anatomy model is a method used to evaluate the case of movement (or lubricity) of devices, such as guidewires or other instruments within the lumen of a catheter, where devices need to be inserted and maneuvered through the catheter without causing damage or discomfort. In some embodiments, this test simulates the conditions that the catheter would experience in the body, including the presence of bodily fluids and the various twists and turns of the vascular system. The goal is to ensure that the catheter's inner surface is sufficiently lubricious to allow smooth, unimpeded movement of devices within it in accordance with some embodiments.

In some embodiments, the catheter is prepared by ensuring it is clean and free of any debris. In some embodiments, a lubricant, often a saline solution or a specific medical-grade lubricant, is applied to the device that will be inserted into the catheter, simulating the presence of bodily fluids. In some embodiments, a traversing structure (e.g., a guidewire) is inserted into the catheter and maneuvered through it, where the case of movement is observed and recorded. This can be done manually or using a machine that can apply a consistent force according to some embodiments. In some embodiments, the force required to move the device through the catheter is measured using a force gauge or similar instrument. The lower the force required to move the traversing structure; the more lubricious the catheter's inner diameter is considered to be in accordance with some embodiments.

As shown in FIG. 21, in some embodiments, track forces for system catheters are significantly less than conventional catheters in similar sections. In some embodiments, a track force for system catheters is less than 0.2 lbs force (lbf) for any catheter section. In some embodiments, a track force for system catheters is less than. 1 lbf force for any catheter section. In some embodiments, a track force for system catheters falls within a range of 0.2 lbf-. 05 lbf force for any catheter section.

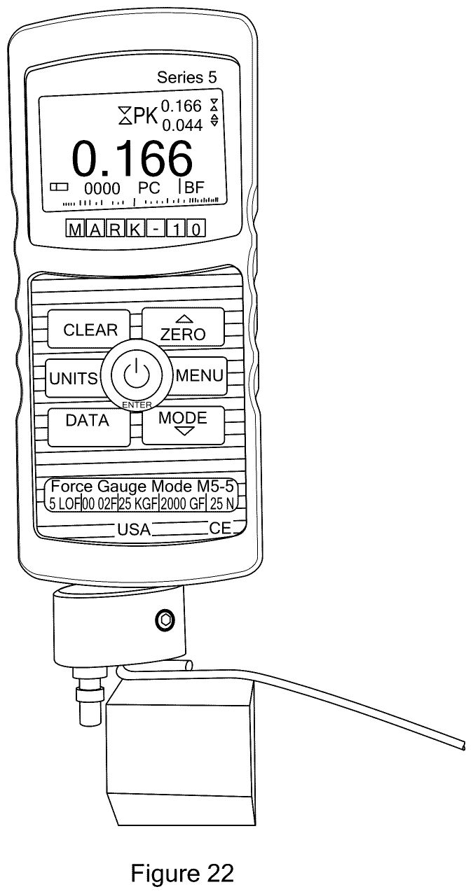

FIG. 22 shows a measured catheter distal looped compression force according to some embodiments. In some embodiments, the catheter is configured to provide a Catheter Distal Looped Compression Force of 0.009-0.300 lbf. In some embodiments, catheter tip spring back force is measured by bending the distal 2 cm to 90 degrees and measuring with a force gage the spring back force as it returns to straight. In some embodiments, spring back force of the distal end (tip) includes a range of 0.001-0.100 lbf.

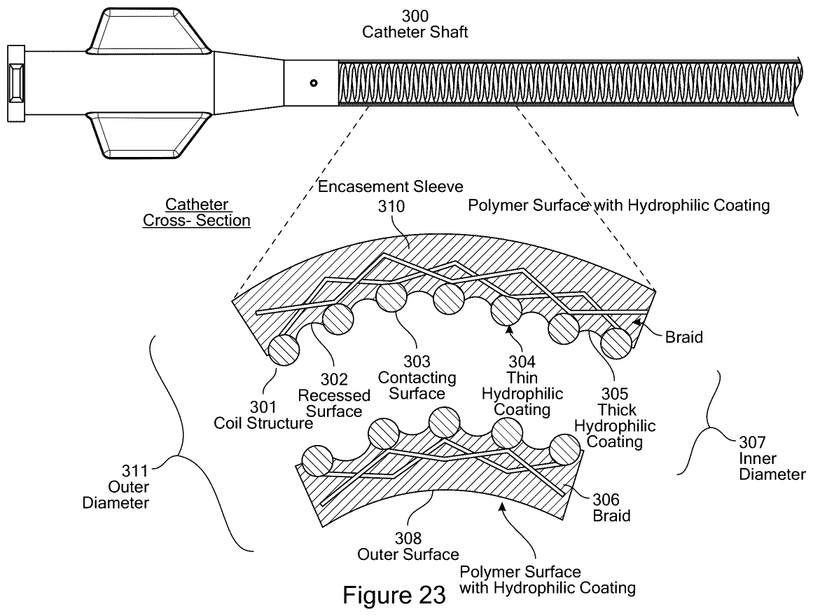

FIG. 23 shows a cross-sectional view of a catheter shaft 300, according to some embodiments. In some embodiments, the catheter shaft 300, which may be a part of any catheter described herein and/or shown in the figures, includes a coil structure 301, which is formed from one or more elements such as wire, string, or laser cut hypotube as previously described in according to some non-limiting embodiments. In some embodiments, the coil structure 301, in combination with an encasement sleeve 310, is configured to form recesses 302 along the inner diameter 307 of the catheter 300. These recessed surface 302 are set back from the contacting surface 303 in the ranges previously described according to some embodiments.

The contacting surface 303, which contacts traversing device surfaces during delivery and/or blood clots during aspiration, includes a thin hydrophilic coating 304 with a thickness from approximately 0.0001″ to 0.000198″ in some embodiments. In some embodiments, the recessed surface 302 includes a thick hydrophilic coating 305 ranging from 0.0002″ to 0.003″.

In some embodiments, the catheter 300 includes a braid 306. In some embodiments, the braid 306 is applied over the coiled structure 301. In some embodiments, only the braid 306 is used, where areas between the braid define the recessed surfaces. The braid 306, which is formed from a selected material, braid pattern, and braid picks per inch (PPI), contributes to the desired structural integrity of the catheter 300.

In some embodiments, the catheter shaft 300 includes an encasement sleeve 310 which may include one or more polymers described herein. Once the encasement sleeve 310 is coupled and/or fused to the reinforcement structure 48, which includes coil structure 301 and/or braid 306 in this non-limiting example, the combination of the reinforcement structure 48 and the encasement sleeve 310 creates peaks in valleys within the inner diameter 307 of the formed catheter shaft 300. In some embodiments, as the encasement sleeve 310 is deformed during the coupling process, at least part of the encasement sleeve 310 flows through the hollow portions formed by the reinforcement structure 48. In some embodiments, once coupled, the catheter shaft 300 includes a smooth outer surface 308 defining the limits of the outer diameter 311 which is smoother than the surface of the inner diameter 307, wherein each surface is coated with at least one hydrophilic coating, enhancing the catheter's lubricity and ease of use during medical procedures.

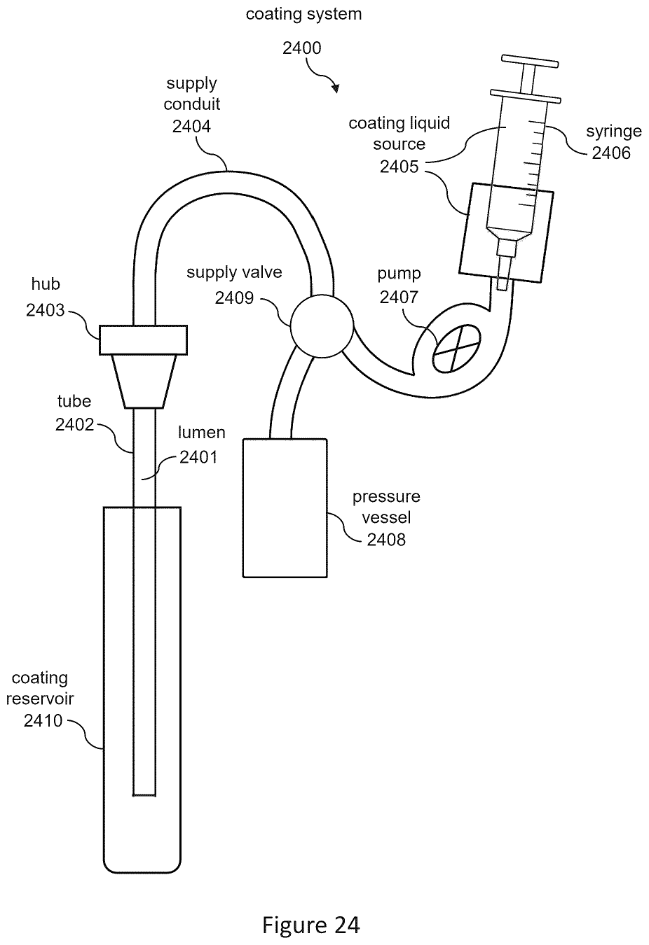

FIG. 24 illustrates a coating system 2400 configured to deliver a coating liquid to a lumen 2401 of a tube 2402 in accordance with some embodiments. In some embodiments, the tube 2402 includes a hub 2403 at a proximal end (as described in relation to other figures), where the hub 2403 is fluidically coupled to a supply conduit 2404. In some embodiments, a supply conduit 2404 couples the hub 2403 to a coating liquid source 2405. In some embodiments, the coating liquid source 2405 includes a reservoir containing one or more coating composition selected for the inner-diameter coating process described herein.

In some embodiments, the coating liquid source 2405 may be coupled to and/or include a syringe 2406 configured to provide positive and/or negative displacement of the coating liquid from the coating liquid source 2405 toward the lumen 2401 via the supply conduit 2404. In some embodiments, the syringe 2406 is replaced with or supplemented by a pump 2407, such as a (bi-directional) piston pump or a peristaltic pump, configured to provide positive and/or negative displacement. In some embodiments, the system 2400 includes a pressure vessel 2408 configured to provide gas-assisted displacement or purge functions during filling and/or draining operations.

In some embodiments, a supply valve 2409 is disposed along the supply conduit 2404 and is configured to direct the coating liquid from the coating liquid source 2405 toward the hub 2403 and to isolate, combine, vent, or switch flow paths during different phases of operation. In some embodiments, the syringe 2406, the pump 2407, the pressure vessel 2408, and the supply valve 2409 are arranged to provide a closed, controllable fluid path that limits air entrainment and facilitates repeatable coating outcomes.

In operation, in some embodiments, the coating system 2400 is configured to fill at least a portion of the lumen 2401 by actuating one or more of the syringe 2406, the pump 2407, or the pressure vessel 2408 to positively displace a measured volume of coating liquid from the coating liquid source 2405 through the supply conduit 2404 and into the tube 2402 via the hub 2403. For example, the syringe 2406 is advanced at a controlled rate until the lumen 2401 is filled to a predetermined length, at which point displacement is paused to allow a dwell period that promotes coating of the inner surface.

In some embodiments, after the dwell period, excess coating liquid is removed from the lumen 2401 to leave a film on the inner surface. In some embodiments, the syringe 2406 and/or the pump 2407 is reversed to withdraw liquid by applying negative pressure. In some embodiments, the pressure vessel 2408 is actuated to apply a controlled gas pressure or vacuum that displaces excess liquid from the lumen 2401. In some embodiments, the lumen 2401 may be drained by gravity, pressure displacement using a gas, mechanical assistance, or any combination of methods described herein, where the draining/displacement of liquid is executed such that a (thin) film of the coating solution remains on the inner surface of the lumen 2401. In some embodiments, the supply valve 2409 is configured to direct the withdrawn liquid back to the coating liquid source 2405 or to a waste container.

In some embodiments, the film remaining on the inner surface of tube 2402 (e.g., catheter shaft 300) is cured to form a uniform coating. In some embodiments, tube 2402 is subjected to a thermal and/or ultraviolet curing regimen (discussed above) appropriate for the selected coating chemistry. In some embodiments, flow or pressure from the pressure vessel 2408 is maintained during curing to stabilize the film and improve thickness uniformity. In some embodiments, a second coating layer is applied using the same positive displacement equipment by repeating the filling, dwell, draining, and curing steps with a second coating liquid from the coating liquid source 2405.

In some embodiments, the coating system 2400 includes a coating reservoir 2410 configured to capture and retain a volume of coating liquid during a coating operation. In some embodiments, the coating reservoir 2410 is positioned to receive coating liquid displaced from the lumen 2401 of the tube 2402 during a positive displacement cycle, reducing material waste and enabling reuse of the coating liquid for subsequent operations.

In some embodiments, the coating reservoir 2410 is configured to facilitate coating of the outer surface of the tube 2402 by immersion. In some embodiments, the tube 2402 is dipped into the coating reservoir 2410 after the reservoir has been filled with coating liquid, such that at least a portion of the outer surface of the tube 2402 is submerged. In some embodiments, the depth of immersion is controlled to achieve a target coating length along the outer surface.

In some embodiments, the coating reservoir 2410 is further configured to serve as a source for coating the inner surface of the tube 2402. In some embodiments, the lumen 2401 is filled by drawing coating liquid from the coating reservoir 2410 through the hub 2403 using negative displacement, such as via the syringe 2406 or the pump 2407. In some embodiments, the coating liquid is displaced into the lumen 2401 before the tube 2402 is dipped into the coating reservoir 2410, so that the inner surface is coated prior to outer surface immersion. In some embodiments, the lumen 2401 is filled after the dipping step, allowing the inner surface to be coated following the outer surface coating, allowing for different exposure times for the different surfaces.

In some embodiments, a method comprises coating the inner and outer surface of the tube 2402 using the coating liquid delivered through the inner diameter of the tube 2402 into the coating reservoir 2410 to fill and surrounding the tube 2402 before removing the tube with the inner and outer surface being coated. In some embodiments, the coating reservoir 2410 is configured to hold the coating solution in such a way that the solution contacts both the inner and outer surfaces of the tube once the container is filled to the desired level. In some embodiments, by filling the coating reservoir 2410 through the tube 2402, excess air bubbles that can occur from dipping the tube into solution can be avoided.

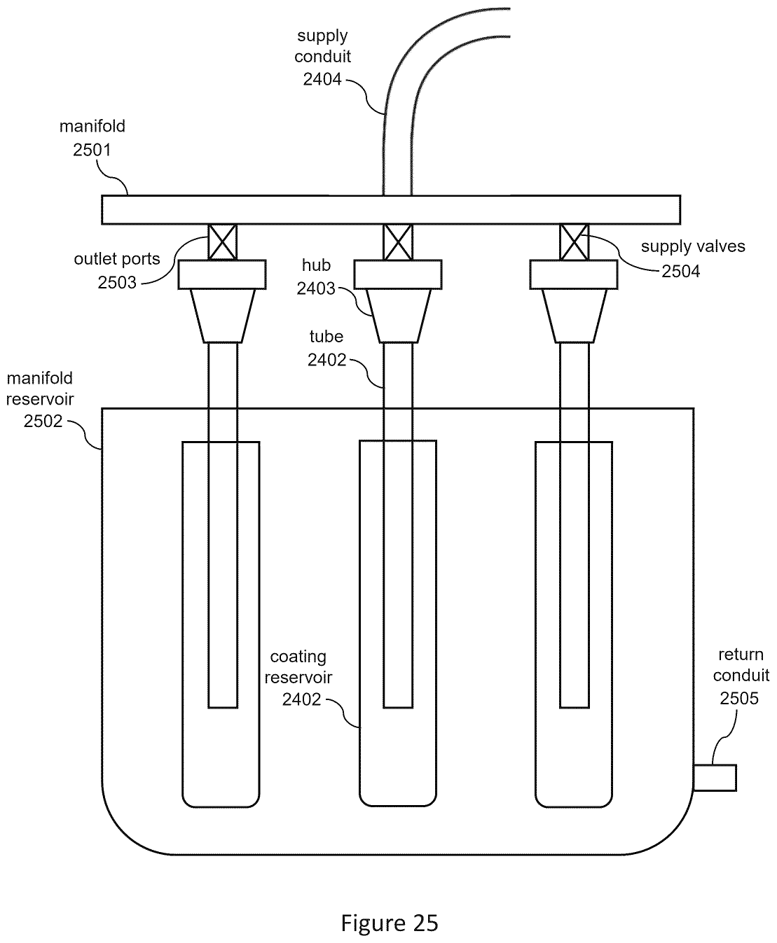

FIG. 25 illustrates a manifold configuration in which a manifold 2501 is fluidically coupled to a manifold reservoir 2502 to support coating of multiple tubes 2402 in parallel. In some embodiments, the manifold 2501 includes a plurality of outlet ports 2503, each configured to couple to a respective hub 2403 of a tube 2402 via a corresponding section of the supply conduit 2404. In some embodiments, the manifold reservoir 2502 is in fluid communication with the coating liquid source(s) 2405 through supply conduit 2404 and is configured to provide a common supply to the manifold 2501.

In some embodiments, one or more supply valves 2509 are provided at or near the ports of the manifold 2501 and are configured to isolate individual branches, balance flow, and sequence filling and draining across multiple tubes 2402. In some embodiments, the syringe 2406, the pump 2407, and/or pressure vessel 2408 is actuated to displace coating liquid from the coating liquid source 2405 through the manifold 2501, with the supply valves 2409 opened in a predetermined order to achieve substantially equivalent fill lengths in each lumen 2401. In some embodiments, negative pressure is supplied to the manifold 2501 via supply conduit 2404 to draw coating liquid into one or more lumens from a coating reservoir 2410 or manifold reservoir 2502.

In some embodiments, the manifold configuration is configured to perform a coordinated dwell period across all connected tubes 2402, after which the system performs a parallel drainage step using one or mor steps described herein. In some embodiments, the syringe 2406 is retracted, the pump 2407 is reversed, or the pressure vessel 2408 is actuated to withdraw or expel excess liquid simultaneously while leaving a film on the inner surface of each lumen 2401. In some embodiments, the manifold reservoir 2501 and or coating reservoir(s) 2410 includes one or more return conduit 2505 configured to couple the manifold reservoir 2502 and/or coating reservoirs 2402 to the coating liquid source 2405 to enable recovery and/or delivery of excess liquid.

In some embodiments, the manifold reservoir 2502 is configured to operate independently of the coating reservoirs 2402 to supply coating liquid for both filling and draining operations. For example, the manifold reservoir 2502 is filled with a predetermined volume of coating liquid and serves as the sole source for coating each connected tube 2402. In some embodiments, the manifold reservoir 2502 is configured to maintain sufficient capacity to accommodate the combined volume required to fill the lumens 2401 of all connected tubes 2402, as well as any excess liquid displaced during the coating process. In some embodiments, the manifold reservoir 2502 is further configured to receive returned coating liquid during a drainage step, enabling recovery and reuse of the coating liquid without the need for individual coating reservoirs 2402. In some embodiments, the manifold reservoir 2502 includes one or more coating reservoirs 2402 in order to allow for limit the amount of fluid needed for single or small batched of tubes.

In some embodiments, the manifold configuration improves throughput and reduces material usage by enabling multiple tubes 2402 to be coated with the same displacement stroke while maintaining control of volume, pressure, and timing at each branch through the supply valves 2409. In some embodiments, uniformity across branches is further enhanced by selecting equal-length sections of the supply conduit 2404 and by using matched hub 2403 geometries to reduce path-to-path variation.

In some embodiments, the manifold configuration is used for sequential multi-layer processes. For example, a first coating liquid is delivered to all connected lumens 2401, drained, and cured, after which the manifold reservoir 2502 is flushed or refilled with a second coating liquid and the displacement sequence is repeated to form a second layer. In some embodiments, process parameters, including displacement rate, dwell time, drainage profile, and cure cycle, are adjusted per layer to achieve target thickness and surface properties.

In some embodiments, the thin film of the coating solution left on the lumen 2401 surface is cured to form a durable and uniform coating. Curing may be achieved through various means, such as heat curing, UV curing, and/or air drying, depending on the nature of the coating solution used, in accordance with some embodiments described herein.

In some embodiments, different coating solutions can be used in one or more coating steps to apply additional and/or different types of layers. In some embodiments, each subsequent layer can be applied by positive displacement, drained, and cured in the same or similar manner as the first coating layer and/or second coating layer as described above. In some embodiments, the system includes one or more multi-functional coatings including hydrophilic, antimicrobial, and/or lubricious layers, where the type and position of the layer depends on the intended application. In some embodiments, supply valve 2409 may be used to connect to different coating liquid sources.

The system and methods described herein, in accordance with some embodiments, offer several advantages. The system limits the amount of wasted coating solution by using a controlled volume of coating solution and provides precise and accurate control over the volume and pressure of the coating solution, allowing for consistent and uniform coating thickness throughout the lumen. In some embodiments, the system is scalable to coat multiple tubes simultaneously by using a manifold system and connecting multiple tubes to a single pump or multiple pumps configured to generate positive pressure for the delivery of fluid.

Some embodiments of the system eliminate or reduce air entrapment by reducing the likelihood of bubbles or air pockets forming in the coating in conventional ways, improving the overall quality and consistency of the coated product. In some embodiments, the system is flexible and can be used to apply a variety of coating materials, including hydrophilic, hydrophobic, antimicrobial, and lubricious coatings, making it suitable for a wide range of applications. In some embodiments, the system improves coating uniformity by pushing the coating solution through the lumen rather than relying on vacuum forces, ensuring more even coverage, especially in longer or more complex tube geometrics.

Some embodiments described herein are directed to coating materials used in the displacement process, which may vary depending on the intended application. In some embodiments, the coating solution includes hydrophilic coatings which include high lubricity and hydrophilic properties. In some embodiments, the coating solution includes polyurethane-based coatings configured to impart flexibility, durability, and chemical resistance. In some embodiments, the coating solution includes polyacrylate coatings that includes high lubricity and hydrophilic properties. In some embodiments, the coating solution includes polysaccharide coatings that include biocompatibility and non-thrombogenic properties. In some embodiments, the coating solution includes antimicrobial coatings for medical devices requiring infection resistance. In some embodiments, the coating materials can be heat-cured, UV-cured, or air-dried depending on the specific formulation, as described above.

In some embodiments, the method comprises providing a first coating solution comprising a mixture of polyaziridine, polyurethane, and/or polyacrylate. In some embodiments, the polyaziridine functions as a crosslinking agent configured to enhance adhesion of the coating to the substrate and to improve chemical and mechanical durability of the resulting film. In some embodiments, the polyurethane is configured to impart flexibility, toughness, and abrasion resistance to the coating layer, thereby maintaining integrity during bending or manipulation of the tube. In some embodiments, the polyacrylate is configured to provide a smooth, uniform film with high lubricity and chemical resistance, improving the overall surface quality of the coated lumen.

In some embodiments, the polyaziridine comprises a crosslinker including one or more of trimethylolpropane tris(2-methyl-1-aziridine propionate) (TMAZ), N,N′-(methylenedi-p-phenylene)bis(aziridine-1-carboxamide) (DAZ), trimethylolpropane bis(2-methyl-1-aziridine propionate), pentaerythritol bis(3-(1-aziridinyl) propionate, pentaerythritol tris(3-(1-aziridinyl) propionate (TAZ), and pentaerythritol tetrakis(3-(1-aziridinyl) propionate.

In some embodiments, the polyaziridine comprises a crosslinker including one or more of trimethylolpropane tris(2-methyl-1-aziridine propionate) (TMAZ), N,N′-(methylenedi-p-phenylene)bis(aziridine-1-carboxamide) (DAZ), and pentacrythritol tris(3-(1-aziridinyl) propionate (TAZ). In some embodiments, the polyaziridine comprises trimethylolpropane tris(2-methyl-1-aziridine propionate) (TMAZ).

In some embodiments, the method comprises the second coating solution additionally comprising a crosslinker. In some embodiments, the second coating solution comprises polysaccharide and/or polyacrylic acid. In some embodiments, the polysaccharide is configured to provide biocompatibility and hydrophilic properties, forming a hydrated surface that reduces friction during device navigation. In some embodiments, the polyacrylic acid is configured to enhance hydrophilicity and wetting characteristics, further improving lubricity and facilitating smooth passage of instruments through the lumen. In some embodiments, the second coating solution is hydrophilic and is configured to absorb water and swell slightly upon hydration, thereby creating a low-friction interface that complements the structural durability provided by the first coating solution.

Once the coating solution has been introduced and the tube 2402 is adequately coated, the tube 2402 is removed from the container. In some embodiments, the tube 2402 is withdrawn from the container. In some embodiments, the coating liquid is withdrawn from the coating reservoir 2410 and the lumen 2401 by holding the tube 2402 in a same position and drawing the solution through the inner lumen 3401 using a vacuum force. This ensures that both the inner and outer surfaces of the tube are uniformly coated. This dual-surface coating method is particularly advantageous for applications where both the internal and external surfaces of the tube require specific functional properties, such as enhanced lubricity, biocompatibility, or antimicrobial characteristics. The ability to coat both surfaces simultaneously also improves process efficiency and ensures consistent coating quality across the entire tube. In some embodiments, the tube comprises a catheter.

Some embodiments include a step of the first coating solution being drained from the lumen by flowing a gas through the lumen. Some embodiments include a step of flowing a cleaning solution through the tube. In some embodiments, the method comprises the first coating solution further comprising a short chain alcohol. In some embodiments, the method comprises the first coating solution additionally comprising a zirconate or titanate compound.

None of the steps described herein are essential or indispensable. Any of the steps according to some embodiments can be adjusted or modified. In some embodiments, other or additional steps can be used. Any portion of any of the steps, processes, structures, and/or devices disclosed or illustrated in some embodiments, flowcharts, or examples in this specification can be combined or used with or instead of any other portion of any of the steps, processes, structures, and/or devices disclosed or illustrated in some embodiments, flowcharts, or examples.

For example, in some embodiments, a method can be described as providing a coating apparatus comprising a tube, a supply conduit, and a fluid displacement component. Some embodiments include a step of positively displacing a coating liquid through the supply conduit into a lumen of the tube using the fluid displacement component. Some embodiments include a step of allowing the coating liquid to remain in contact with an inner wall of the tube for a predetermined amount of time. Some embodiments include a step of draining the coating liquid from the lumen.

In some embodiments, the coating apparatus further comprises a coating liquid source configured to contain the coating liquid. In some embodiments, the coating liquid source is coupled to the supply conduit. In some embodiments, the coating liquid source includes a syringe internal volume. In some embodiments, the fluid displacement component includes a syringe.

Some embodiments include a step of providing a coating reservoir. Some embodiments include a step of positively displacing the coating liquid through the lumen into the coating reservoir until the coating reservoir is filled to a level where at least a quarter of a length of an outer surface of the tube has been coated by the coating liquid. Some embodiments include a step of withdrawing the tube from the coating reservoir after the predetermined amount of time.

In some embodiments, draining the coating liquid from the lumen includes draining the coating liquid as the tube is withdrawn from the coating reservoir. Some embodiments include a step of withdrawing the coating liquid from the coating reservoir by applying a negative pressure to the tube. Some embodiments include a step of providing a manifold. Some embodiments include a step of delivering the coating liquid from the supply conduit to a plurality of tubes via the manifold.

A method may also be described as negatively displacing a coating liquid through the supply conduit into a lumen of the tube using the fluid displacement component. Some embodiments include a step of allowing the coating liquid to remain in contact with an inner wall of the tube for a predetermined amount of time. Some embodiments include a step of removing the coating liquid from the lumen.

In some embodiments, the fluid displacement component includes a pump. In some embodiments, the coating liquid source includes a coating reservoir. Some embodiments include a step of drawing the coating liquid from the coating reservoir into the lumen using negative pressure. Some embodiments include a step of drawing the coating liquid from the coating reservoir by applying the negative pressure to the tube while the tube is being inserted into the coating reservoir. Some embodiments include a step of drawing the coating liquid into the lumen until at least a quarter of the inner wall is coated.

Some embodiments include a step of providing a manifold. Some embodiments include a step of coupling a plurality of tubes to the manifold. Some embodiments include a step of drawing the coating liquid into the plurality of tubes using the negative pressure.

It is understood that the system is not limited in its application to the details of construction and the arrangement of components set forth in the previous description or illustrated in the drawings. The system and methods disclosed herein fall within the scope of numerous embodiments. The previous discussion is presented to enable a person skilled in the art to make and use the system according to some embodiments. Any portion of the structures and/or principles included in some embodiments can be applied to any and/or all embodiments: it is understood that features from some embodiments presented herein are combinable with other features according to some other embodiments. Thus, some embodiments of the system are not intended to be limited to what is illustrated but are to be accorded the widest scope consistent with all principles and features disclosed herein.

Some embodiments of the system are presented with specific values and/or setpoints. These values and setpoints are not intended to be limiting and are merely examples of a higher configuration versus a lower configuration and are intended as an aid for those of ordinary skill to make and use the system.

Any text in the drawings is part of the system's disclosure and is understood to be readily incorporable into any description of the metes and bounds of the system. Any functional language in the drawings is a reference to the system being configured to perform the recited function, and structures shown or described in the drawings are to be considered as the system comprising the structures recited therein. It is understood that defining the metes and bounds of the system using a description of images in the drawing does not need a corresponding text description in the written specification to fall with the scope of the disclosure.

Furthermore, acting as Applicant's own lexicographer, Applicant imparts the explicit meaning and/or disavow of claim scope to the following terms:

Applicant defines any use of “and/or” such as, for example, “A and/or B,” or “at least one of A and/or B” to mean element A alone, element B alone, or elements A and B together. In addition, a recitation of “at least one of A, B, and C,” a recitation of “at least one of A, B, or C,” or a recitation of “at least one of A, B, or C or any combination thereof” are each defined to mean element A alone, element B alone, element C alone, or any combination of elements A, B and C, such as AB, AC, BC, or ABC, for example.

“Substantially” and “approximately” when used in conjunction with a value encompass a difference of 5% or less of the same unit and/or scale of that being measured (e.g., degrees, volume, mass, distance).

As used herein, “can” or “may” or derivations thereof are used for descriptive purposes only and is understood to be synonymous and/or interchangeable with “configured to” when defining the metes and bounds of the system.

The terms “can” or “may” may also be used in place of “in some embodiments” or derivations thereof to emphasis the modularity of the system where different components may be excluded or included based on a desired result without departing from the scope of the system as a whole, which can be defined using any combination of features described herein.

In addition, the term “configured to” means that the limitations recited in the specification and/or the claims must be arranged in such a way to perform the recited function: “configured to” excludes structures in the art that are “capable of” being modified to perform the recited function but the disclosures associated with the art have no explicit teachings to do so. For example, a recitation of a “container configured to receive a fluid from structure X at an upper portion and deliver fluid from a lower portion to structure Y” is limited to systems where structure X, structure Y, and the container are all disclosed as arranged to perform the recited function. The recitation “configured to” excludes elements that may be “capable of” performing the recited function simply by virtue of their construction but associated disclosures (or lack thereof) provide no teachings to make such a modification to meet the functional limitations between all structures recited.

It is understood that the phraseology and terminology used herein is for description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The previous detailed description is to be read with reference to the figures, in which like elements in different figures have like reference numerals. The figures, which are not necessarily to scale, depict some embodiments and are not intended to limit the scope of embodiments of the system.

It will be appreciated by those skilled in the art that while the system has been described above in connection with some embodiments and examples, the system is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the system are set forth in the following claims.

I claim:

1. A method comprising:
providing a coating apparatus comprising a tube, a supply conduit, and a fluid displacement component;
positively displacing a coating liquid through the supply conduit into a lumen of the tube using the fluid displacement component;
allowing the coating liquid to remain in contact with an inner wall of the tube for a predetermined amount of time; and
draining the coating liquid from the lumen.

2. The method of claim 1,
wherein the coating apparatus further comprises a coating liquid source configured to contain the coating liquid.

3. The method of claim 2,
wherein the coating liquid source is coupled to the supply conduit.

4. The method of claim 3,
wherein the coating liquid source includes a syringe internal volume.

5. The method of claim 4,
wherein the fluid displacement component includes a syringe.

6. The method of claim 1, further including steps of:
providing a coating reservoir; and
positively displacing the coating liquid through the lumen into the coating reservoir until the coating reservoir is filled to a level where at least a quarter of a length of an outer surface of the tube has been coated by the coating liquid.

7. The method of claim 6, further including a step of:
withdrawing the tube from the coating reservoir after the predetermined amount of time.

8. The method of claim 7, further including a step of:
where draining the coating liquid from the lumen includes draining the coating liquid as the tube is being withdrawn from the coating reservoir.

9. The method of claim 6, further including a step of:
withdrawing the coating liquid from the coating reservoir by applying a negative pressure to the tube.

10. The method of claim 1, further comprising steps of:
providing a manifold; and
delivering the coating liquid from the supply conduit to a plurality of tubes via the manifold.

11. A method comprising:
providing a coating apparatus comprising a tube, a supply conduit, and a fluid displacement component;
negatively displacing a coating liquid through the supply conduit into a lumen of the tube using the fluid displacement component;
allowing the coating liquid to remain in contact with an inner wall of the tube for a predetermined amount of time; and
removing the coating liquid from the lumen.

12. The method of claim 11,
wherein the coating apparatus further comprises a coating liquid source configured to contain the coating liquid.

13. The method of claim 12,
wherein the coating liquid source is coupled to the supply conduit.

14. The method of claim 13,
wherein the coating liquid source includes a syringe internal volume.

15. The method of claim 14,
wherein the fluid displacement component includes a syringe.

16. The method of claim 12,
wherein the fluid displacement component includes a pump.

17. The method of claim 12, further including a step of:
wherein the coating liquid source includes a coating reservoir; and
further including a step of drawing the coating liquid from the coating reservoir into the lumen using negative pressure.

18. The method of claim 17, further comprising a step of:
drawing the coating liquid from the coating reservoir by applying the negative pressure to the tube while the tube is being inserted into the coating reservoir.

19. The method of claim 17, further including a step of:
drawing the coating liquid into the lumen until at least a quarter of the inner wall is coated.

20. The method of claim 19, further including a step of:
providing a manifold;
coupling a plurality of tubes to the manifold; and
drawing the coating liquid into the plurality of tubes using the negative pressure.
